(19) United States
(12) Patent Application Publication
(10) Pub. No.: US 2003/0059872 A1
(43) Pub. Date: Mar. 27, 2003

(54) Nucleic acids, expression vectors and host cells for making chimeric nucleic acids and methods for producing immobilized polypeptides

(75) Inventors: Hiroko Abe, Ibaraki (JP); Yoh-ichi Shimma, Ibaraki (JP); Yoshifumi Jigami, Ibaraki (JP)

Correspondence Address: FISH & RICHARDSON P.C., Suite 500, 4350 La Jolla Village Drive, San Diego, CA, 92122, US

(73) Assignee: National Institute of Advanced Industrial Science and Technology

(21) Appl. No.: 09/989,975

(22) Filed: Nov. 21, 2001

(30) Foreign Application Priority Data
Nov. 21, 2000 (JP) 2000-354396
Jun. 22, 2001 (JP) 20001-190524

(51) Int. Cl. C07H 21/04; C12N 9/10; C12N 1/18; C12P 21/02

(52) U.S. Cl. 435/69.1; 435/320.1; 435/193; 435/254.2; 536/23.2

(57) ABSTRACT

The present invention enables immobilization of a useful protein, for example, a glycosyltransferase, onto the surface of a yeast cell without deteriorating its enzyme activity. The present invention provides a fusion nucleic acid, expression vectors and host cells comprising these chimeric nucleic acids, and methods for making and using them. The chimeric nucleic acid is characterized by comprising a coding sequence encoding a useful protein bound to a yeast cell wall protein. In one aspect, it is downstream of a gene encoding the yeast cell wall protein. In one aspect, the host cell is a transformant yeast, which is transformed with the fusion gene expression vector that expressed an enzyme that is immobilized yeast cell wall.

### RELATED APPLICATIONS

This application is a continuation in part (CIP) and claims the benefit of priority under 35 U.S.C. §119 to Japan patent application no. 2000-354396, filed Nov. 21, 2000; and Japan patent application no. 20001-190524, filed Jun. 22,2001. The aforementioned applications are explicitly incorporated herein by reference in its entirety and for all purposes.

### TECHNICAL FIELD

The present invention generally relates to the fields of biochemistry, molecular biology and protein synthesis. In particular, the invention is directed to fusion nucleic acid sequences, expression vectors and transformed host cells comprising a coding sequence for a Pir (protein internal repeat) protein and a peptide or polypeptide of interest. The invention is also directed to chimeric polypeptides comprising a Pir (protein internal repeat) protein and a peptide or polypeptide of interest, such as a useful enzyme. The invention is also directed to methods for making and using these chimeric nucleic acids, expression vectors and host cells.

### BACKGROUND

An enzyme protein used for producing substances with a bioreactor or the like is generally used as an immobilized enzyme which is immobilized to an insoluble carrier. This is for the sake of the convenience of procedures and from an economical standpoint. A general method for producing an immobilized enzyme involves purifying enzyme proteins and immobilizing the proteins to resin beads or the like. However, mass purification of enzyme proteins may not only involve complex procedures, but also may cost a great deal, and cause inactivation of enzyme activity during a purification process. Further, a process to immobilize to a carrier, e.g., resin beads, may also inactivate enzyme activity. There are methods which produce an enzyme protein using it as an immobilized enzyme inside an intact cell. However, in these cases, the efficacy of enzyme reaction cannot be said to be optimum since the enzyme protein is located within a cell. Hence, it is desired to localize an enzyme protein on the surface of a cell that is a carrier.

One of the binding patterns of plasma membrane proteins to a membrane involves addition of the glycolipid, GPI (glycosylphosphatidylinositol), to the C-terminus of a protein which is anchored to a membrane by the lipid portion of GPI (GPI anchor) embedded within the membrane (Conzelmann, EMBO J. 7, 2233-2240, (1988)). Immobilization of a cell wall protein (GPI-anchored protein) having the GPI anchor onto a cell wall comprises cleavage and removal of the C-terminal portion of a protein in the endoplasmic reticulum during the protein biosynthesis, addition of a GPI anchor to the protein, modification of a GPI core sugar chain in the Golgi apparatus, transportation to a cell membrane, transfer of the GPI glycan to a cell wall glucan, and covalent attachment to the cell wall has been reported (see, e.g., Lu, J. Cell, Biol. 128, 333-340, (1995)). Thus, a GPI anchor is a useful means to immobilizea protein to the surface layer of a cell. For example, an established technique employs alpha-agglutinin that is anchored by GPI to a cell wall to immobilize a foreign protein to a cell wall (see, e.g., Schreuder et al., Trends Biotechnol., 14, 115-120, (1996)). However, a defect of GPI anchors is that a GPI anchor functions for localization and immobilization to a cell wall only when a GPI attachment signal and the C-terminus of a protein are fused, and does not function when the signal and the C-tertninus are not fused (see, e.g., Lipke, Mol. Cell Biol. 9, 3155-3165, (1989)). As a method to avoid such drawback, a modified method of immobilization to a cell surface layer with a GPI anchor has been reported which employs association of two subunits composing an a-agglutinin, AGAlp (see, e.g., Roy, Mol Cell Biol., 11, 4196-4206, (1991)) and AGA2p (Cappellaro, EMBO J., 10, 4081-4088, (1991)) on the surface layer of a cell (see, e.g., Boder and Wittrup, Nature Biotechnol. 15, 553-557, 1997). In this method, a fusion protein (to which a target protein has been linked to the C-terminal side of AGA2p) is bound to AGAlp (which has been immobilized on a cell wall by a GPI anchor) via S-S bonding on the cell wall so as to immobilize the target protein to the cell surface layer. However, this method can be applied only to a cell expressing AGA1p.

On the other hand, human hormones, most physiologically active substances and the receptors of these physiologically active substances consist of proteins and sugar chains. The sugar chain portion plays an important role in physiological activity, and a protein body alone cannot exhibit its original function (see, e.g., Akira Kobata, Protein, Nucleic Acid and Enzyme, 36, 775-788 (1991)). A glycosyltransferase is an essential enzyme in the formation of a sugar chain structure. It is thought that there are several hundred types of human glycosyltransferase. There must therefore be an enormous number of different types of glycosyltransferases when those of microorganisms and plants are included. A glycosyltransferase possesses an extremely high substrate specificity so that it adds a certain sugar to a receptor sugar chain with a certain structure by a certain binding pattern, thereby synthesizing a sugar chain with a certain structure (see, e.g., Qwens, Biochem. Biophys. Res. Commun., 109, 1075-1082, (1982); Betteridge, Eur. J. Biochem., 132, 29-35, (1983)). That is, the structure of a sugar chain is infinitely diversified such that a variety of sugars are associated by various binding patterns including branching. The diversity of the structure is determined by the combination of the substrate specificities of various glycosyltransferases. Accordingly, production and utilization of complex carbohydrates requires efficient expression and preparation of a glycosyltransferase that synthesizes a necessary sugar chain structure, because a substance functioning in vivo cannot be produced without precise control of the sugar chain structure.

Most glycosyltransferases are type II membrane proteins, and are localized over the membrane of the Golgi apparatus with a topology in which an active region on the C-terminal side is oriented to the lumen of the Golgi apparatus (Paulson, J. Biol. Chem., 264, 17615-17618, 1989). Therefore, when the C-terminal side of a glycosyltransferase is genetically altered, the enzyme activity will often be deteriorated. It is very difficult to immobilize a glycosyltransferase onto a cell wall while maintaining its enzyme activity, using the above-described GPI anchor that cannot finction unless it is bound to the C- terminal side. Meanwhile, the N-terminal side of a glycosyltransferase contains a transmembrane region, by which the enzymes are localized to ER or Golgi membrane. Soluble glycosyltransferase forms are also present in biological fluid. They arise from proteolytic cleavage in the stem region. That is, addition of a novel anchor protein (for immobilization to cell surface layer) to a glycosyltransferase lacking a transmembrane region would be effective in controlling localization of glycosyltransferases.

A Pir (protein with internal repeat) protein is a cell wall protein covalently bound to a yeast cell wall, and Pir 1 to 4 genes, which are homologous to each other, compose a family (TOH-E, YEAST 9:481-494, (1993); Mrsa, YEAST 13:1145-1154, (1997)). However, a Pir protein has no GPI-anchor attachment signal. Further, since Pir proteins are not eluted with a detergent, but break away from a cell wall under alkali conditions, they may be localized and bound to a cell wall by a binding mechanism different from those of GPI-anchored proteins and other non-covalently proteins, and their mechanism remains unknown (Mrsa (1999) YEAST 15:813-820).

There has been an attempt to use Pir as an anchor protein, in which a protein A gene is inserted between genes encoding Pir 4 proteins to be localized on a cell wall (Moukadiri et al., J. Bacteriology, 181, 4741-4740, (1999)). However the binding pattern to a cell wall is so varied among Pir proteins that Pir proteins cannot be generalized. For example, Pir4 can be freed from a cell wall by b-mercaptoethanol treatment, while Pir1 and Pir2 cannot be freed by the same treatment but can be eluted from a cell wall only under alkali conditions. Therefore, Pir 4 is thought to differ from other Pirl, Pir2 and Pir3, in the binding mechanism to a cell wall (Moukadiri et al., J. Bacteriology, 181, 4741-4740, (1999)). Further, a protein fused to Pir4 is a protein A, which does not require a very precise structure compared to a protein, for example, an enzyme, and is not fused to the N-terminal side of a target protein. Hence, when a protein to be expressed is, for example, an enzyme protein, has activity closely related to its structure, use of a gene encoding a Pir4 protein as an anchor protein does not always cause the resulting, expressed protein to retain enzyme activity.

### SUMMARY OF THE INVENTION

The invention provides a chimeric nucleic acid comprising a first domain comprising a yeast Pir cell wall protein coding sequence and a second domain comprising a peptide or a polypeptide coding sequence, wherein the yeast cell wall protein is capable of being localized or immobilized on a yeast cell wall. In one aspect of the chimeric nucleic acid, the yeast cell wall protein comprises a Pir (protein internal repeat) cell-wall binding motif coding sequence. The Pir (protein internal repeat) protein motif can comprise an amino acid sequence as set forth by SEQ ID NO:1 or SEQ ID NO:2, or, the Pir (protein internal repeat) protein motif can comprise a protein comprising an amino acid sequence derived from an amino acid sequence as set forth by SEQ ID NO:1 or SEQ ID NO:2 by deletion, replacement, or addition of one or more amino acids of SEQ ID NO:1 or SEQ ID NO:2, wherein the Pir (protein internal repeat) protein motif is capable of being localized or immobilized on a yeast cell wall.

In one aspect of the chimeric nucleic acid, the peptide or a polypeptide coding sequence fused to the yeast cell wall protein can comprise all or part of an enzyme, e.g., a catalytic domain of an enzyme. The enzyme can be any glycosyltransferase. The peptide or a polypeptide coding sequence fused to the yeast cell wall protein can be any peptide or a polypeptide, such as a receptor, an antibody, a bioluminescent marker, and the like.

In one aspect of the chimeric nucleic acid, the yeast cell wall protein coding sequence, e.g., the Pir (protein internal repeat) protein motif, is located 5′ to the peptide or a 3 polypeptide coding sequence. Thus, when the chimeric polypeptide is expressed, the yeast cell wall binding motif is amino terminal to the peptide or a polypeptide of interest. However, the yeast cell wall protein can be located anywhere in the chimeric polypeptide.

The invention provides an expression cassette comprising a chimeric nucleic acid comprising a first domain comprising a yeast cell wall protein coding sequence, such as a Pir (protein internal repeat) protein motif, and a second domain comprising a peptide or a polypeptide coding sequence. In one aspect, the expression cassette is an expression vector, such as a yeast expression vector.

The invention provides a host cell comprising an expression cassette comprising a chimeric nucleic acid comprising a first domain comprising a yeast cell wall protein coding sequence, such as a Pir (protein internal repeat) protein motif, and a second domain comprising a peptide or a polypeptide coding sequence. In one aspect, the host cell is a microorganism, such as a yeast cell, or, any microorganism comprising yeast cell wall.

The invention provide an expression vector comprising a fusion gene comprising a nucleic acid encoding a useful protein downstream of a nucleic acid encoding a yeast cell wall protein selected from the group consisting of (a) a protein having an amino acid sequence represented by SEQ ID NO:1 or SEQ ID NO:2, and (b) a protein comprising an amino acid derived from an amino acid sequence as set forth by SEQ ID NO:1 or SEQ ID NO:2 by deletion, replacement, or addition of one or more amino acids of SEQ ID NO: 1 or SEQ ID NO:2, wherein yeast cell wall protein is capable of being localized or immobilized on a yeast cell wall. The useful protein can be any peptide or polypeptide, such as an enzyme, e.g., a glycosyltransferase protein.

The invention provide a transformant yeast transformed by an expression vector, wherein the expression vector comprises a chimeric nucleic acid comprising a nucleic acid encoding a useful protein downstream of a nucleic acid encoding a yeast cell wall protein selected from the group consisting of (a) a protein having an amino acid sequence represented by SEQ ID NO:1 or SEQ ID NO:2, and (b) a protein comprising an amino acid derived from an amino acid sequence as set forth by SEQ ID NO: 1 or SEQ ID NO:2 by deletion, replacement, or addition of one or more amino acids of SEQ ID NO: 1 or SEQ ID NO:2, wherein yeast cell wall protein is capable of being localized or immobilized on a yeast cell wall.

The invention provide a chimeric polypeptide comprising a first domain comprising a yeast cell wall protein and a second domain comprising a peptide or a polypeptide of interest, wherein the yeast cell wall protein is capable of being localized or immobilized on a yeast cell wall.

The invention provide a particle comprising a chimeric polypeptide comprising a first domain comprising a yeast cell wall protein and a second domain comprising a peptide or a polypeptide of interest, wherein the yeast cell wall protein is capable of being localized or immobilized on a yeast cell wall component, and a yeast cell wall component. The particle can be any material, e.g., a resin.

The invention provide a solid support comprising a chimeric polypeptide comprising a first domain comprising a yeast cell wall protein and a second domain comprising a peptide or a polypeptide of interest, wherein the yeast cell wall protein is capable of being localized or immobilized on a yeast cell wall component, and a yeast cell wall component. The solid support can comprise any material or configuration, e.g., a tube, a fiber, a plate or a filter.

The invention provide a method for producing an immobilized polypeptide comprising the following steps: (a) providing an expression vector, wherein the expression vector comprises a chimeric nucleic acid encoding a fusion polypeptide, wherein the chimeric nucleic acid comprises a nucleic acid encoding a useful protein downstream of a nucleic acid encoding a yeast cell wall protein selected from the group consisting of (a) a protein having an amino acid sequence represented by SEQ ID NO: 1 or SEQ ID NO:2, and (b) a protein comprising an amino acid derived from an amino acid sequence as set forth by SEQ ID NO: 1 or SEQ ID NO:2 by deletion, replacement, or addition of one or more amino acids of SEQ ID NO: 1 or SEQ ID NO:2, wherein yeast cell wall protein is capable of being localized or immobilized on a yeast cell wall; (b) transforming a microorganism comprising a yeast cell wall with the expression vector of step (a); (b) culturing the transformant microorganism of step (b) and expressing the fusion polypeptide on a surface layer of the yeast cell wall, thereby producing an immobilized polypeptide. In one aspect, the useful protein is an enzyme, e.g., a glycosyltransferase protein. The microorganism can comprise a yeast.

The invention provides an immobilized enzyme obtained by a method of the invention. The immobilized enzyme of the invention, wherein the enzyme is a glycosyltransferase.

The invention provides a method for producing a sugar chain or a sugar comprising use of an immobilized enzyme of the invention.

The invention provides a method for producing an immobilized enzyme comprising culturing the host cell of the invention and obtaining a yeast comprising a useful protein immobilized on its cell wall.

The invention provides an immobilized enzyme obtained by the method of the invention. The immobilized enzyme of the invention, wherein the enzyme immobilized is a glycosyltransferase.

The invention provides a method for producing a sugar chain or a sugar that employs the immobilized enzyme of the invention.

The invention provides a transformant yeast that is transformed by allowing the yeast to comprise an expression cassette of the invention or an expression vector of the invention.

The invention provides a method for producing an immobilized enzyme which comprises the steps of: (a) culturing the transformant yeast of the invention, (b) expressing chimeric polypeptides on the surface layer a cell wall of the transformant yeast, and (c) isolating a transformant yeast that expresses a chimeric polypeptide immobilized on the cell wall.

The invention provides an immobilized enzyme obtained by the method of the invention. he immobilized enzyme of the invention, wherein the enzyme immobilized is a glycosyltransferase.

The invention provides a method for producing a sugar chain or a sugar, wherein the method comprises sequentially converting a sugar chain or a sugar using an immobilized enzyme of the invention.

The invention provides a chimeric nucleic acid comprising a first domain comprising a yeast cell wall protein coding sequence and a second domain comprising an enzyme coding sequence, wherein the yeast cell wall protein is capable of being localized or immobilized on a yeast cell wall and the enzyme is selected from the group consisting of a fucosyltransferase, a Lacto-N-fucopentaose, a galactosyltransferase, and a glucosyltransferase.

The invention provides a chimeric polypeptide comprising a first domain comprising a yeast cell wall protein and a second domain comprising an enzyme, wherein the yeast cell wall protein is capable of being localized or immobilized on a yeast cell wall and the enzyme is selected from the group consisting of a fucosyltransferase, a Lacto-N- fucopentaose, a galactosyltransferase, and a glucosyltransferase.

The purpose of the present invention is to localize and immobilize a protein, such as an enzyme, e.g., a glycosyltransferase on a surface, such as a yeast cell wall. In particular, the present invention is to localizes and immobilizes a glycosyltransferase, which is deteriorated by genetic manipulation of the C-terminus, on the surface layer of a yeast cell wall while maintaining its activity, and providing the protein as an immobilized enzyme. As a result of thorough studies to solve the above problems, the inventors found that a fusion protein comprising a Pir protein, or structurally and functionally related polypeptides, bound to the N-terminus of a useful protein can be expressed on the surface layer of a yeast cell wall while maintaining the activity of the useful protein. The protein can be expressed on the surface layer of a yeast cell wall by transformation of yeast with a fusion gene expression vector. The vector contains the chimeric, or fusion, nucleic acid, or gene, that comprises a sequence encoding the useful protein bound downstream of a gene encoding the Pir protein of a yeast cell wall.

The invention provides a fusion gene expression vector which contains a fusion gene comprising a gene encoding a useful protein bound downstream of a gene encoding the following yeast cell wall protein (a) or (b): (a) a protein having an amino acid sequence represented by SEQ ID NO: 1 or SEQ ID NO:2; (b) a protein having an amino acid sequence derived from the amino acid sequence represented by SEQ ID NO: 1 or SEQ ID NO:2 by deletion, replacement, or addition of one or more amino acids, and having ability to be localized and immobilized on a yeast cell wall. In the fusion gene expression vector, the useful protein can be a glycosyltransferase protein.

The invention provides a transformant yeast which is transformed by the fusion gene expression vector of the invention. The invention provides a method for producing an immobilized enzyme, which comprises culturing the transformant yeast of the invention, expressing a fusion gene on the surface layer of a yeast cell wall, and obtaining yeast that contains a useful protein immobilized on the cell wall. The invention provides an immobilized enzyme, which is obtained by the method of the invention. The immobilized enzyme of the invention can be immobilized is a glycosyltransferase.

The invention provides a method for producing a sugar chain or sugars which method employs the immobilized enzyme of the invention.

The invention provides a fusion gene expression vector which contains a fusion gene comprising a gene encoding a useful protein bound downstream of a gene encoding the following yeast cell wall protein (a) or (b): (a) a protein having an amino acid sequence represented by SEQ ID NO: 2; (b) a protein having an amino acid sequence derived from the amino acid sequence represented by SEQ NO: 2 by deletion, replacement, or addition of one or more amino acids, and having ability to be localized and immobilized on a yeast cell wall. In the fusion gene expression vector, the useful protein can be a glycosyltransferase protein. The invention provides a transformant yeast, which is transformed with this fusion gene expression vector.

The invention provides a method for producing an immobilized enzyme wherein the method comprises culturing the transformant yeast of the invention, expressing a fusion gene on the surface layer of a yeast cell wall, and obtaining yeast that contains a useful protein immobilized on the cell wall.

The invention provides an immobilized enzyme, which is obtained by the method of the invention. The immobilized enzyme can be immobilized is a glycosyltransferase. The invention provides a method for producing a sugar chain or sugars which method employs the immobilized enzyme of the invention. The invention provides a transformant yeast, wherein the yeast is transformed by allowing the yeast to contain at least two or more types of the fusion gene expression vector of the invention.

The invention provides a method for producing an immobilized enzyme, wherein the method comprises culturing the transformant yeast of the invention, expressing simultaneously fusion genes on the surface layer of a yeast cell wall, and obtaining yeast that contains at least two or more types of useful proteins immobilized on the cell wall. The invention provides an immobilized enzyme, which is obtained by the method of the invention. The immobilized enzyme can be immobilized are two or more types of glycosyltransferases. The invention provides a method for producing a sugar chain or sugars which method sequentially converts sugar chains or sugars using the immobilized enzymes of the invention.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

FIG. 1 is a schematic diagram showing the structure of the construct YEp352GAP-II(PIR1-HA-gma12)(pAB4).

FIG. 2 is a representation of a photograph showing the results of detecting expression of a fusion protein for a transformant strain W303-YEp352GAP-II(PIR1-HA- gma12) and a control strain W303-YEp352GAP-II by the indirect immunofluorescence technique.

FIG. 3 is a graphic summary showing the results of detecting galactosyltransferase activity for a transformant strain W303-YEp352GAP-II(PIR1-HA- gma12) and a control strain W303-YEp352GAP-II. The tip of an arrow in the figure indicates a peak of galactosyl mannobiose.

FIG. 4 is a schematic diagram showing the structure of the construct YEp352GAP-II(PIR1-HA-FUT6)(pAB9).

FIG. 5 is a representation of a photograph showing the results of detecting expression of a fusion protein for a transformant strain W303-YEp352GAP-II (PIR1-HA- FUT6) and a control strain W303-YEp352GAP-II.

FIG. 6 is a schematic diagram showing results of detecting fucosyltransferase activity for a transformant strain W303-YEp352GAP-II (PIR1-HA-FUT6) and a control strain W303-YEp352GAP-II. The tip of an arrow in the figure indicates a peak of Lacto-N-fucopentaose.

FIG. 7 is a schematic diagram showing the structure of the construct YEp352GAP-II(PIR1-HA-KRE2).

FIG. 8 is a schematic diagram showing the structure of the construct YEp352GAP-II(PIR2-FLAG-MNN1).

FIG. 9 simultaneous expression of YEp352GAP-II(PIR1-HA-KRE2) and YEp351GAP-II(PIR2-FLAG-MNN1) in transformant yeast.

FIG. 10 is a schematic representation of radiographs of PAGE gels indicating that a yeast strain W303-YEp352GAP-II(PIR1-HA-KRE2), YEp351GAP-ll(PIR2-FLAG-MNN1) which has been transformed with Pir1-HA-Kre2 and Pir2-FLAG-Mnn1 at the same time performed sequential transfer reaction of mannose resulting from the two expression vectors YEp352GAP-II (PIR1-HA-KRE2) and YEp351GAP-II(PIR2-FLAG-MNN1).

### DETAILED DESCRIPTION

The present invention provides novel compositions and methods for localization and immobilization of compositions on a yeast cell wall. In one aspect, the invention is directed to a fusion, or chimeric, nucleic acid sequence comprising a coding sequence for a Pir (protein internal repeat) protein and a peptide or polypeptide of interest. Alternative aspects include expression vectors comprising this chimeric nucleic acid sequence and host cells comprising the fusion sequence, e.g., yeast transformed with the expression vector. In one aspect, the Pir motif coding sequence is 5′ to the coding sequence of the protein of interest such that in the expressed chimeric protein the Pir motif is located at the N-terminus of the protein.

In one aspect, the invention is directed to a fusion, or chimeric, polypeptide sequence comprising a coding sequence for a Pir (protein internal repeat) protein and a peptide or polypeptide of interest. The Pir (protein internal repeat) protein motif has the ability to be localized and immobilized on a yeast cell wall. When part of a fusion protein, it enables the chimeric polypeptide to be localized and immobilized on a yeast cell wall. In one aspect, the Pir motif is bound to the N-terminus of a desired, useful protein. In one aspect, the Pir motif bound to the N-terminus of a desired, useful protein.

The desired protein can be an enzyme, an antibody or a binding protein, e.g., a ligand, such as a receptor. The desired protein can bind to another composition, including polypeptides, polysaccharides, lipids or any small molecule. For example, in one aspect, the immobilized protein is an enzyme immobilized on a yeast cell wall. In one aspect, the immobilized protein is a glycosyltransferase.

In one aspect, the invention provides a method for producing a sugar chain or sugars using a glycosyltransferase immobilized on a yeast cell wall. The yeast is transformed with an expression vector; and an immobilized enzyme comprising glycosyltransferase is immobilized on the yeast cell wall. Thus, the invention provides a method for producing a sugar chain or sugars using a glycosyltransferase immobilized on the above yeast cell wall.

DEFINITIONS

Unless defmed otherwise, all technical and scientific terms used herein have the meaning commonly understood by a person skilled in the art to which this invention belongs. As used herein, the following terms have the meanings ascribed to them unless specified otherwise.

The term “antibody” or “Ab” includes both intact antibodies having at least two heavy (H) chains and two light (L) chains inter-connected by disulfide bonds and antigen binding fragments thereof, or equivalents thereof, either isolated from natural sources, recombinantly generated or partially or entirely synthetic. Examples of antigen binding fragments include, e.g., Fab fragments, F(ab')2 fragments, Fd fragments, dAb fragments, isolated complementarity determining regions (CDR), single chain antibodies, chimeric antibodies, humanized antibodies, human antibodies made in non-human animals (e.g., transgenic mice) or any form of antigen binding fragment.

The term “expression cassette” as used herein refers to a nucleotide sequence which is capable of affecting expression of a structural gene (i.e., a protein coding sequence) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid that can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and nonexpression plasmids. Where a recombinant microorganism, e.g., a yeast cell, or a cell culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The term “nucleic acid” or “nucleic acid sequence” refers to a deoxy-ribonucleotide or ribonucleotide oligonucleotide, including single- or double-stranded forms, and coding or non-coding (e.g., “antisense” ) forms. The term encompasses nucleic acids containing known analogues of natural nucleotides. The term also encompasses nucleic- acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methyl- phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The term nucleic acid is used interchangeably with gene, DNA, RNA, cDNA, mRNA, oligonucleotide primer, probe and amplification product.

As used herein the terms “polypeptide,” “protein,” and “peptide” are used interchangeably and include compositions of the invention that also include “analogs,” or “conservative variants” and “mimetics” (e.g., “peptidomimetics”) with structures and activity that substantially correspond to the polypeptides used with the compositions and the methods of the invention. Thus, the terms “conservative variant” or “analog” or “mimetic” also refer to a polypeptide or peptide which has a modified amino acid sequence, such that the change(s) do not substantially alter the polypeptide's (the conservative variant's) structure and/or activity (e.g., glycosyltransferase activity), as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing finctionally similar amino acids are well known in the art. For example, one exemplary guideline to select conservative substitutions includes (original residue followed by exemplary substitution): ala/gly or ser; arg/lys; asn/gin or his; asp/glu; cys/ser; gln/asn; gly/asp; gly/ala or pro; his/asn or gIn; ile/leu or val; leu/ile or val; lys/arg or gIn or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E);3) Asparagine (N), Glutamine (Q);4) Arginine (R), Lysine (K);5) Isoleucine (I), Leucine (L), Methionine (M), Valine (V); and 6) Phenylalanine (F), Tyrosine (Y), Tryptophan (W); (see also, e.g., Creighton (1984) Proteins, W.H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.” The terms “mimetic” and “peptidomimetic” refer to a synthetic chemical compound that has substantially the same structural and/or functional characteristics of the polypeptides used in the compositions and the methods of the invention (e.g., glycosyltransferase activity). The mimetic can be either entirely composed of synthetic, non- natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics'structure and/or activity. As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond” ) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond” ) linkages include, e.g., ketomethylene (e.g., -C(=O)-CH2- for -C(=O)-NH-), aminomethylene (CH2-NH), ethylene, olefin (CH=CH), ether (CH2-O), thioether (CH2-S), tetrazole (CN4-), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY). A polypeptide can also be characterized as a mimetic by containing all or some non-natural residues in place of naturally occurring amino acid residues; non-natural residues are well described in the scientific and patent literature.

As used herein, the term “glycosyltransferase” can mean any enzyme capable of modifying or synthesizing a polysaccharide, e.g., a fucosyltransferase, a Lacto-N-fucopentaose, a galactosyltransferase, a glucosyltransferase, a mannosyltransferase, a galactosamyltransferase, a sialyltransferase and a N-acetylglucosaminyltransferase. The structure and function of glycosyltransferases, methods of isolating (purification) and making and using glycosyltransferases, nucleic acid and amino acid sequences of glycosyltransferases are well known in the art, see e.g., U.S. Pat. Nos. RE37,206; 6,291,219; 6,270,987; 6,238,894; 6,204,431; 6,143,868; 6,087,143; 6,054,309; 6,027,928; 6,025,174; 6,025,173; 5,955,282; 5,945,322; 5,922,540; 5,892,070; 5,876,714; 5,874,261; 5,871,983; 5,861,293; 5,859,334; 5,858,752; 5,856,159; 5,545,553.

As used herein, the terms “Pir (protein with internal repeat) protein” and “Pir (protein with internal repeat) motif” means a cell wall protein capable of binding to a yeast cell wall that is encoded by a Pir gene, including a Pir 1, Pir 2, Pir3 or Pir 4 gene, or a related member of this genus, which are structurally and functionally related to each other; this genus is a family of yeast cell wall binding proteins, see, e.g., TOH-E, YEAST 9:481-494, (1993); Mrsa, YEAST 13:1145-1154, (1997). The terms also include proteins comprising an amino acid sequence based on, i.e., derived from or designed from, an amino acid sequence as set forth by SEQ NO: 1 by deletion, replacement, or addition of one or more amino acids of SEQ NO: 1, wherein the Pir (protein internal repeat) protein motif is capable of being localized or immobilized on a yeast cell wall. In alternative aspects, the Pir (protein internal repeat) protein comprises an sequence having at least about 70% or more, at least about 75% or more, at least about 80% or more, at least about 85% or more, at least about 90% or more, at least about 95% or more, at least about 97% or more, at least about 98% or more, at least about 99% or more sequence identify, or homology, with the amino acid sequence of SEQ ID NO: 1 or SEQ ID NO: 2 when the sequence identify, or homology, is calculated, e.g., with BLAST, and, the Pir (protein internal repeat) protein retains its yeast cell wall binding properties.

Which amino acid residues can be deleted, added replaced or modified can be determined by methods routine in the art; it is simply a matter of determining a motif or a modification, a deletion, a replacement, or an addition that increases or changes in some desirable way the binding of the peptide or polypeptide to a yeast cell wall component.

Polypeptides and Peptides

The invention provides a chimeric polypeptide comprising a first domain comprising a Pir yeast cell wall binding protein (e.g., SEQ ID NO:1 or SEQ ID NO:2) and a second domain comprising a peptide or a polypeptide of interest, wherein the yeast cell wall binding protein is capable of being localized or immobilized on a yeast cell wall. The polypeptide of interest can be any polypeptide, e.g., an enzyme, e.g., a glycosyltransferase, such as a fucosyltransferase, a Lacto-N-fucopentaose, a galactosyltransferase, a glucosyltransferase, a mannosyltransferase, a galactosamyltransferase, a sialyltransferase and a N-acetylglucosaminyltransferase. Polypeptides and peptides of the compositions and methods of the invention can be isolated in whole or in part from natural sources, be synthetic, or be recombinantly generated polypeptides. Peptides and proteins can be recombinantly expressed in vitro or in vivo. The peptides and polypeptides of the invention can be made and isolated using any method known in the art.

Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A.K., Therapeutic Peptides and Proteins, Formulation, Processing and Delivery Systems (1995) Technomic Publishing Co., Lancaster, PA. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer). The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

The invention provides a fusion protein comprising a polypeptide of interest and a peptide or polypeptide capable of specifically binding to a yeast cell wall component. Peptides and polypeptides of the invention also can be synthesized and expressed as chimeric or “fusion” proteins with additional domains linked thereto for, e.g., to more readily isolate or identify a recombinantly synthesized peptide, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle WA). The inclusion of a cleavable linker sequences such as Factor Xa or enterokinase (Invitrogen, San Diego CA) between the purification domain and GCA-associated peptide or polypeptide can be useful to facilitate purification. For example, an expression vector can include an epitope-encoding nucleic acid sequence linked to six histidine residues followed by a thioredoxin and an enterokinase cleavage site (see, e.g., Williams (1995) Biochemistry 34:1787-1797; Dobeli (1998) Protein Expr. Purif. 12:404-14). The histidine residues facilitate detection and purification while the enterokinase cleavage site provides a means for purifying the epitope from the remainder of the fusion protein.

Nucleic acids expression vectors and transformed cells The invention provides fusion, or chimeric, nucleic acids comprising a first domain comprising a yeast cell wall protein coding sequence and a second domain comprising a peptide or a polypeptide coding sequence, wherein the yeast cell wall protein is capable of being localized or immobilized on a yeast cell wall. In one aspect, the yeast cell wall protein comprises a Pir (protein internal repeat) protein motif coding sequence. The Pir (protein internal repeat) protein motif can comprise an amino acid sequence as set forth by SEQ ID NO:1 or SEQ ID NO:2, or variations thereof. As the genes and vectors of the invention can be made and expressed in vitro or in vivo, the invention provides for a variety of means of making and expressing these genes and vectors. One of skill will recognize that desired phenotypes associated with altered gene activity can be obtained by modulating the expression or activity of the genes and nucleic acids (e.g., promoters) within the expression cassettes (e.g., vectors) of the invention. Any of the known methods described for increasing or decreasing expression or activity can be used for this invention. The invention can be practiced in conjunction with any method or protocol known in the art, which are well described in the scientific and patent literature.

The nucleic acid sequences of the invention and other nucleic acids used to practice this invention, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to insect and bacterial cells, e.g., mammalian, yeast or plant cell expression systems.

Alternatively, these nucleic acids can be synthesized in vitro by well-known chemical synthesis techniques, as described in, e.g., Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The invention provides chimeric nucleic acids of the invention “operably linked” to a transcriptional regulatory sequence. “Operably linked” refers to a finctional relationship between two or more nucleic acid (e.g., DNA) segments. Typically, it refers to the finctional relationship of a transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. For example, in one embodiment, a promoter is operably linked to a chimeric nucleic acid sequence of the invention.

The invention further provides cis-acting transcriptional regulatory sequences, which, in vivo, are operably linked to the coding sequence for the exemplary chimeric polypeptide of the invention, including promoters, comprising the genomic sequences 5′ (upstream) of a transcriptional start site and intronic sequences. The promoters of the invention contain cis-acting transcriptional regulatory elements involved in message expression. These promoter sequences may be readily obtained using routine molecular biological techniques. Genomic sequence can be readily identified by “chromosome walking” techniques, as described by, e.g., Hauser (1998) Plant J 16:117-125; Min (1998) Biotechniques 24:398-400. Other useful methods for further characterization of promoter sequences include those general methods described by, e.g., Pang (1997) Biotechniques 22:1046-1048; Gobinda (1993) PCR Meth. Applic. 2:318; Triglia (1988) Nucleic Acids Res. 16:8186; Lagerstrom (1991) PCR Methods Applic. 1:111; Parker (1991) Nucleic Acids Res. 19:3055. As is apparent to one of ordinary skill in the art, these techniques can also be applied to identify, characterize and isolate any genomic or cis-acting regulatory sequences corresponding to or associated with the nucleic acid and polypeptide sequences of the invention.

The invention provides oligonucleotide primers that can amplify all or any specific region within a nucleic acid sequence of the invention, e.g., SEQ ID NO: 1. The nucleic acids of the invention can also be mutated, detected, generated or measured quantitatively using amplification techniques. Using the nucleic acid sequences of the invention (e.g., SEQ ID NO: 1), the skilled artisan can select and design suitable oligonucleotide amplification primers. Amplification methods are also known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y.); ligase chain reaction (LCR) (see, e.g., Barringer (1990) Gene 89:117); transcription amplification (see, e.g., Kwoh (1989) Proc. Natl. Acad. Sci. USA, 86:1173); and, self-sustained sequence replication (see, e.g., Guatelli (1990) Proc. Natl. Acad. Sci. USA, 87:1874); Q Beta replicase amplification (see, e.g., Smith (1997) J. Clin. Microbiol. 35:1477-1491; Burg (1996) Mol. Cell. Probes 10:257-271) and other RNA polymerase mediated techniques (e.g., NASBA, Cangene, Mississauga, Ontario).

Expression vectors capable of expressing the nucleic acids and polypeptides of the invention in any cell, including yeast cells, bacterial cells, insect cells and mammalian cells, are well known in the art. Vectors which may be employed include recombinantly modified enveloped or non-enveloped DNA and RNA viruses, e.g., from baculoviridiae, parvoviridiae, picornoviridiae, herpesveridiae, poxviridae, adenoviridiae, picornnaviridiae or alphaviridae.

Yeast expression vectors, transcriptional regulatory systems, e.g., promoters and enhancers, yeast cell culture methods and the like are well known in the art, see, e.g., published U.S. patent application 20010012630; U.S. Pat. Nos. RE37,343; 6,312,923; 6,306,625; 6,300,065; 6,258,566; 6,172,039; 6,165,738; 6,159,705; 6,114,147; 6,100,042; 6,083,723; 6,027,910; 5,876,951; 5,739,029; 5,602,034; 5,482,835; 5,302,697.

Insect cell expression systems commonly use recombinant variations of baculoviruses and other nucleopolyhedrovirus, e.g., Bombyx mori nucleopolyhedrovirus vectors (see, e.g., Choi (2000) Arch. Virol. 145:171-177). For example, Lepidopteran and Coleopteran cells are used to replicate baculoviruses to promote expression of foreign genes carried by baculoviruses, e.g., Spodopterafrugiperda cells are infected with recombinant Autographa californica nuclear polyhedrosis viruses (AcNPV) carrying a heterologous, e.g., a human, coding sequence (see, e.g., Lee (2000) J. Virol. 74:11873-11880; Wu (2000) J. Biotechnol. 80:75-83).

Mammalian expression vectors can be derived from adenoviral, adeno-associated viral or retroviral genomes. Retroviral vectors can include those based upon murine leukemia virus (see, e.g., U.S. Pat. No. 6,132,731), gibbon ape leukemia virus (see, e.g., U.S. Pat. No. 6,033,905), simian immuno-deficiency virus, human immuno-deficiency virus (see, e.g., U.S. Pat. No. 5,985,641), and combinations thereof. Describing adenovirus vectors, see, e.g., U.S. Pat. Nos. 6,140,087; 6,136,594; 6,133,028; 6,120,764. See, e.g., Okada (1996) Gene Ther. 3:957-964; Muzyczka (1994) J. Clin. Invst. 94:1351; U.S. Pat. Nos. 6,156,303; 6,143,548 5,952,221, describing AAV vectors. See also 6,004,799; 5,833,993.

The invention provides a transformed cell comprising a nucleic acid of the invention. The cells can be yeast (e.g., yeasts belonging to the genera Candida, Debaryomyces, Hansenula, Kluyveromyces, Pichia and Saccharomyces), mammalian (such as mouse or human), insect (such as Spodopterafrugiperda, Spodoptera exigua, Spodoptera littoralis, Spodoptera litura, Pseudaletia separata, Trichoplusia ni, Plutella xylostella, Bombyx mori, Lymantria dispar, Heliothis virescens, Autographica californica and other insect cell lines), plant, bacterial, and the like. Techniques for transforming and culturing cells are well described in the scientific and patent literature; see, e.g., Weiss (1995) Methods Mol. Biol. 39:79-95, describing insect cell culture in serum-free media; Tom (1995) Methods Mol. Biol. 39:203-224; Kulakosky (1998) Glycobiology 8:741-745; Altmann (1999) Glycoconj. J. 16:109-123; Yanase (1998) Acta Virol. 42:293-298; U.S. Pat. Nos. 6,153,409; 6,143,565; 6,103,526.

In one aspect, a fusion gene expression vector of the present invention contains a fusion gene which comprises a gene of a desired enzyme protein bound downstream of a gene encoding a Pir protein that is present on a yeast cell wall having an amino acid sequence of SEQ ID NO: 1 or 2.

PIR genes used in the present invention include a gene (PIRI) encoding a Pirl protein represented by SEQ ID NO: 1, and a gene (PIR2) encoding a Pir2 protein represented by SEQ ID NO: 2, and a gene encoding a protein having an amino acid sequence derived from the amino acid sequence above by deletion, replacement or addition of one or more amino acids and having ability to be localized or immobilized to a yeast cell wall.

Examples of an amino acid sequence derived from the amino acid sequence represented by SEQ ID NO: 1 or 2 by deletion, replacement or addition of one or more amino acids include: an amino acid sequence which is derived from the amino acid sequence of SEQ ID NO: 1 or 2 by deletion of one to 10 amino acids, e.g., 1 to 5 amino acids, or 1 to 2 amino acids; an amino acid sequence which is derived from the amino acid sequence of SEQ ID NO: 1 or 2 by replacement of one to 10 amino acids, e.g., 1 to 5 amino acids, or 1 to 2 amino acids by other amino acids; and an amino acid sequence which is derived from the amino acid sequence of SEQ ID NO: 1 or 2 by addition of one to 10 amino acids, e.g., 1 to 5 amino acids, or 1 to 2 amino acids. Examples of such an amino acid sequence designed, based on, or derived from the amino acid sequence represented by SEQ ID NO:1 or SEQ ID NO:2 by deletion, replacement or addition of one or more amino acids have at least 80% or more, 90% or more, 95% or more, 97% or more, 98% or more, 99% or more homology with the amino acid sequence of SEQ ID NO: 2 when homology is calculated with BLAST. Such an amino acid sequence designed, based on or derived from the amino acid sequence represented by SEQ ID NO: 1 or SEQ ID NO:2 by deletion, replacement or addition of one or more amino acids is substantially identical to the amino acid sequence of SEQ ID NO: 1 or SEQ ID NO:2.

More specifically, these genes can be obtained by PCR using a genome obtained from budding yeast (Saccharomyces cerevisiae) strain W303-1A (ura3, leu2, his3, trp1, ade2) (see, e.g., Kainuma et al., Glycobiology Vol.9, 133-141(1999)) as a template. A primer used herein contains a restriction enzyme cleavage portion, which is useful for easy cleavage of a portion encoding a Pir protein and simple insertion of a epitope tag sequence and the like.

Any desired, useful protein gene may be bound downstream of the above PIR gene. For example, a gene of a protein such as a glycosyltransferase whose activity may be deteriorated by gene manipulation of the C-terminus, is preferred. Examples of such a protein include a galactosyltransferase, fucosyltransferase, glucosyltransferase, mannosyltransferase, galactosamyltransferase, sialyltransferase and N-acetylglucosaminyltransferase.

In one aspect, an expression cassette is constructed by inserting a promoter to upstream and a terminator to downstream of a fusion gene comprising a desired protein gene bound downstream of a PIR gene. Then, the expression cassette is introduced into an vector. Alternatively, when a promoter and a terminator have already been present in an expression vector into which the fusion gene is introduced, only the fusion gene is introduced between the promoter and the terminator without constructing any expression cassette.

For a promoter in an expression cassette, any promoter which is generally used for a yeast expression system and allows expression of a fusion gene introduced in a transformed yeast cell can be used. Examples of such a promoter include, but are not specifically limited to, include PGK, GAP, TPI, GALl, GALlO, ADH2, PH05 and CUP1. For a terminator, any terminator which is generally used for a yeast expression system and allows termination of transcription when it is located downstream of a fusion gene introduced can be used. Examples of such a terminator include ADH1, TDH1, TFF and TRP5.

Examples of an expression vector to which an expression cassette is introduced are not specifically limited as long as they are generally used in a yeast expression system, and allow expression of a fusion gene on the surface layer of the cell wall of transformant yeast that has been transformed with the expression vector. For example, a yeast episome expression vector can be used. A yeast episome plasmid vector contains a 2m plasmid sequence, which is an original sequence of yeast and the vector is rendered capable of replication within a host yeast cell using the autonomously replicating sequence of the 2m plasmid sequence. Examples of a yeast episome expression vector used in the present invention are not specifically limited, as long as they contain at least an ARS sequence of the yeast 2 m plasmid sequence and they can replicate outside the chromosome within a host yeast cell. Such a yeast episome expression vector may be YEp5 l, pYES2, YEp351, YEp352 or the like.

The above yeast episome expression vector is preferably a shuttle vector, which can proliferate within E. coli cells to perform subcloning in recombinant E. coli. A more preferred expression vector contains a selective marker gene, such as an ampicillin-resistant gene. In addition, the expression vector contains a marker gene with which a yeast clone can be selected based on auxotrophy and drug resistance when recombinant yeast is produced. Examples of a marker gene include HIS3, TRP1, LEU2, URA3, ADE2, CAN1, SUC2, LYS2, and CUP1 (Yasuji Oshima (writer-editor), Experimental Protocols in Biochemistry 39, Experimental Protocols in Yeast Molecular Genetics, 119-144 (1996)). These are merely examples, and selection should be made depending on a genotype of a yeast strain to be used as a host for gene transfer.

The above series of techniques involved in construction of a fusion gene expression plasmid may be appropriately performed by persons skilled in the art by referring to descriptions given in examples described later or by standard technology.

In this invention, examples of host yeast to be transformed with the above fusion gene expression vector include, but are not limited to, yeast belonging to the genus Saccharomyces and the genus Candida. Examples of yeast belonging to the genus Saccharomyces include Saccharomyces cerevisiae strains KK4, Y334, Inv-Scl and W303.

To transform yeast with a fusion gene expression vector, for example, known methods such as a lithium acetate method, electroporation, and the like can be used (see, e.g., Becker and Guarente, Methods Enzymol., 194, 182-187 (1991)).

In the present invention, yeast may be transformed simultaneously using multiple expression vectors in which genes encoding different useful proteins are bound to the same type of PIR genes. Yeast may also be transformed simultaneously using multiple expression vectors in which genes encoding different useful proteins are bound respectively to the different types of PIR genes (e.g. PIR1 gene and PIR2 gene). In these cases, for example, by using as genes encoding useful proteins multiple genes (different, but related to each other) encoding glycosyltransferase proteins, immobilized enzymes of the transformed yeast can perform multiple reactions sequentially. Hence, these cases have an advantage that greatly diverse sugar chains or sugars can be produced. Here, the term “immobilized enzyme” means an enzyme immobilized to a yeast cell wall.

An appropriate selective marker is used for screening for transformant yeast. In a preferred example, a gene involved in the metabolism on a chromosomal DNA of a host cell is used. That is, when transforming a host cell having the above gene on chromosomal DNA which has been disabled function by appropriate techniques, e.g., mutant with an expression vector that contains a corresponding normal gene, a preferred selection marker is one which can be used to screen by proliferating the transformant cell that contains a normal metabolism gene. More specifically, a widely used selective marker gene, for example, URA3, and LEU2 as described above is integrated to an expression vector. For a chromosome incorporation type (YIp type), these genes are also used as markers for screening.

Culturing of the transformed transformant yeast enables expression of a fusion protein comprising a Pir protein bound to the N-terminus of a desired protein onto the surface layer of the cell wall. The desired protein is immobilized via Pir on the surface layer of the cell wall of the transformant yeast, so that the transformant yeast can be used directly as an immobilized enzyme. Culturing of transformant yeast can be performed by standard techniques for culturing yeast.

A medium used herein contains a yeast assimilable carbon source, a nitrogen source, inorganic salts and the like to enable efficient culturing of transformants. For example, a synthetic medium that can be used herein (containing a carbon source, a nitrogen source, inorganic salts, amino acids, vitamins and the like) contains various medium components (supplied from Difco) added thereto, except amino acid (required for replication and maintenance of plasmids, but can be supplied with a marker) which is removed from the medium (Sherman, Methods Enzymol., 194, 3-57 (1991)). pH for a medium is preferably adjusted to 6 to 8. Adjustment of pH is performed using inorganic or organic acid, alkali solution, urea, calcium carbonate, ammonia or the like. Culturing is performed at about 28° C. to about 32° C., e.g., at 30° C., for 15 to 48 hours with aeration and agitation.

It will be readily apparent to one skilled in the art that various substitutions and modifications may be made to the invention disclosed herein without departing from the scope and spirit of the invention. It is understood that the examples and aspects described herein are for illustrative purposes only and that various modifications or changes in light thereof will be suggested to persons skilled in the art and are to be included within the spirit and purview of this application and scope of the appended claims.

### EXAMPLES

The following examples are offered to illustrate, but not to limit the claimed invention.

Example 1: Cloning of budding yeast PIR1 gene

PIR1 gene was isolated by PCR using the genome obtained from a budding yeast (Saccharomyces cerevisiae) strain W303-1A (ura3, leu2, his3, trp1, ade2) (Kainuma et al., Glycobiology Vol. 9:133 (1999)) as a template.

A primer was designed based on a base sequence which has been registered on a database (DB name: GenBank; Accession NO: D13740). At this time, a primer previously containing an SacI site at the N-terminal portion and an NotI site at the C-terminal portion was so designed that a portion encoding a protein can be easily cleaved out with a restriction enzyme and a epitope tag sequence and the like can be simply inserted. Base sequences for each primer are as follows.

| | | |
|---|---|---|
| Forward primer: | 5′-GGGGGGAGCTCATGCAATACAAAAAATCATTAGTTGCCTCCGCC-3′ | (SEQ ID NO: 3) |
| Reverse primer: | 5′-CCCCCGCGGCCGCACAGTGCAAATCGATAGC-3′ | (SEQ ID NO: 4) |

The underlined portion in the base sequence of the forward primer indicates the Sacl site; and that of the reverse primer indicates the NotI site. Thus, the designed primers were synthesized by standard techniques.

Composition of the PCR solution is as shown in Table 1. Composition of PCR solution

TABLE 1

| Composition of PCR solution | |
|---|---|
| 10 × EXPAND ™ buffer | 10 μl |
| (including 15 mM MgCl₂) | |
| dNTP Mixture (2.5 mM each) | 8 μl |
| Forward primer (20 pmpl/μl) | 2 μl |
| Reverse primer (20 pmpl/μl) | 2 μl |
| Genome DNA | 3 μl |
| EXPAND ™ High Fidelity PCR | 0.75 μl |
| System enzyme mix | |
| Water | 74.25 μl |
| Total | 100 μl |

The first PCR reaction consisted of 10 cycles. The temperature conditions for each cycle consisted of denaturation of template DNA at 94° C. for 2 min, 94° C for 15 sec (denaturation), 50° C. for 30 sec (annealing), and 72° C. for 1 min (elongation). The next reaction consisted of 15 cycles, the temperature conditions for each cycle consisting of 94° C. for 15 sec (denaturation), 50° C. for 30 sec (annealing), and 72° C. for 1 min (elongation) and each cycle having a prolongation of 5 sec. Finally, elongation reaction at 72° C. was performed for 7 min. The amplified DNA fragment with a length of approximately lkbp obtained by the PCR was cleaved with NotI-SacI, and then inserted to an NotI-SacI site of pBluescript II SK(-) (Stratagene), thereby constructing pBSII (PIR1) (pAB2).

Example 2: Construction of fusion protein composed of fission yeast Gmal12 and HA Tag A gma12 gene (DB name: GenBank; Accession No: Z30917) (Chappell, Mol. Biol. Cell, 5, 519-528 (1994))of fission yeast (Schizosaccharomyces pombe) was also cloned by PCR. Composition of the PCR solution is shown in Table 2.

TABLE 2

| Composition of PCR solution | |
|---|---|
| 10 × EXPAND ™ buffer (including | 10 μl |
| 15 mM MgCl₂) | |
| dNTP Mixture (2.5 mM each) | 8 μl |
| Forward primer (20 pmpl/μl) | 2 μl |
| Reverse primer (20 pmpl/μl) | 2 μl |
| Plasmid DNA | 1 μl |
| EXPAND ™ High Fidelity PCR System | 0.75 μl |
| enzyme mix | |
| Water | 76.25 μl |
| Total | 100 μl |

The plasmid (pYD1-HA-gma12) that has been constructed from gma12 gene cloned into YEpU-GAP-gma12 (YOKO-O et al., FEBS, Vol.257, 1998) by Takayama who belonged to the inventors'laboratory was used as a template. A primer was so designed to enable amplification of HA-gma12 fusion gene that the amplified product previously contains an NotI site on the 5′side and an SmaI site on the 3′side. Primers having the following base sequences were used.

| | |
|---|---|
| 5′-GGGGGGCGGCCGCATACCCATACGATGTTCCTGAC | (SEQ ID NO: 5) |
| Reverse primer: | |
| 5′-GGGGCCCGGGCTAGGATGATGGTTTCAAAAGATTTTGAATATGATCC | (SEQ ID NO: 6) |

The underlined portion in the base sequence of the forward primer indicates the NotI site; that of the reverse primer indicates the Smal site. PCR was performed under temperature conditions the same as those employed for amplification of PIR1 by PCR.

Subsequently, the HA-gma12 fusion gene was inserted to an NotI-Smal site of pBluescript II SK(-) (Stratagene), thereby constructing pBSII (HA-gmal2) (pAB1).

Example 3: Preparation of PIR1-HA-gmal2 fusion gene, fusion gene expression vector, and transformant yeast containing the plasmid A PIRI gene inserted in pBSII(PIRl) was cleaved with SacI-NotI, and then inserted into an SacI-NotI site of pBSII (HA-gmal2), thereby constructing pBSII (PIR1-HA- gma12) (pAB3).

A PIR1-HA-gma12 portion of the pBSII (PIR-HA-gma12) was cleaved with SacI-Smal. Then the product was inserted into an SacI-Smal site of a expression vector YEp352GAP-II (Nakayama) in which the multi-cloning site of a yeast expression vector YEp352GAP (Roy et al., J. Biol. Chem., Vol.237, 2538 (1998)) has been replaced by a portion from EcoRI to SalI of the multi-cloning site of pUC18, thereby constructing YEp352GAP-II(PIR1-HA-gma12) (pAB4) (FIG.1).

The expression vector YEp352GAP-II(PIR1-HA-gma12) was transformed to a yeast strain W303-1A (ura3, leu2, his3, trp1, ade2) (Kainuma et al., Glycobiology, 9□133-141(1999)), thereby obtaining a strain W303- YEp352GAP-II(PIR1-HA-gma12).

In addition, the strain W303- YEp352GAP-II(PIR1-HA-gma12) was deposited under the Accession No. FERM BP-7794 at the International Patent Organism Depository, National Institute of Advanced Industrial Science and Technology on November 16, 2000.

Example 4: Expression of Pir1-HA-Gma12 fusion protein within yeast cells The transformant obtained in Example 3 was examined by the indirect immunofluorescence technique to ascertain whether a fusion gene was expressed within a cell and a fusion protein was presented on the surface layer of the yeast cell.

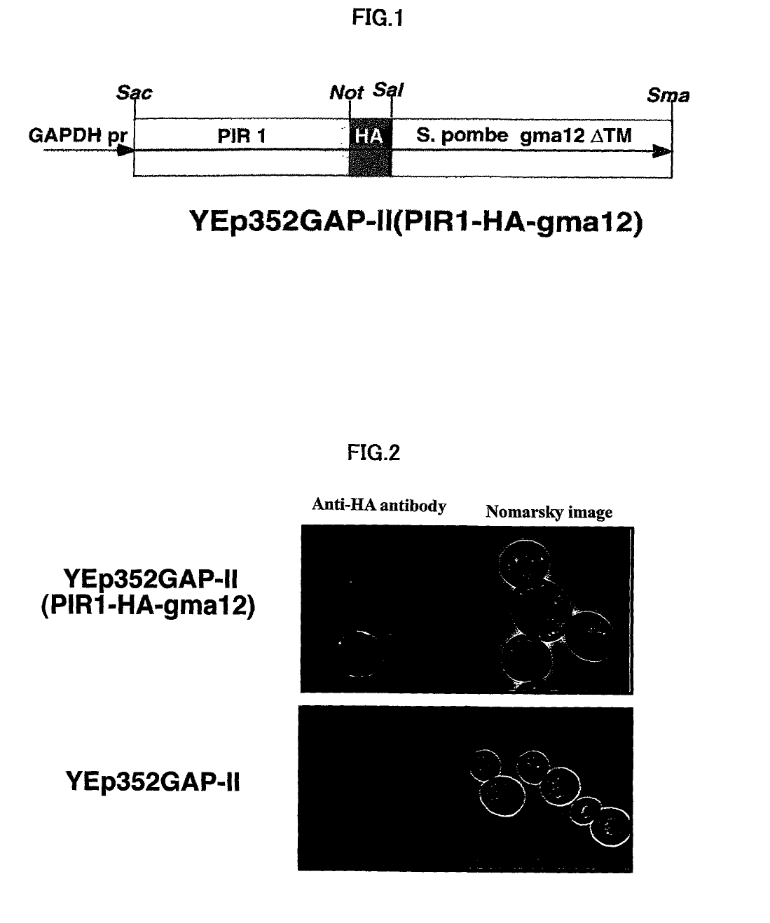

First, the above transformant strain W303-YEp352GAP-II(PIR1-HA-gma12) and a control strain W303-YEp352GAP-II, which had been transformed from a strain W303-1A using YEp352GAP-II, were cultured in 5ml of a SD (-uracil) liquid medium up to OD600=5 (for approximately 30 hours). Then, 1 ml of the culture solution was collected, and then the cells were washed with PBS; (8mg/ml NaCl, 0.2mg/ml KC1, 1.44 mg / ml Na₂HPO₄, 0.24 mg/ml KH2PO₄ (pH 7.4) ). The cells were collected, suspended in 250 ml of a PBS solution □8 mg/ml NaCl, 0.2 mg/ml KCl,1.44 mg/ml Na₂HPO₄, 0.24 mg/ml KH₂PO₄ (pH7.4), 1 mg/ml BSA) containing 1 mg of HA antibody [Anti-HA High Affinity (Roche)], and then incubated on ice for 30 min. The cells were collected and washed once with a PBS solution. Subsequently, the cells were suspended in 250ml of a PBS solution (8 mg/ml NaCl, 0.2 mg/ml KCl, 1.44 mg/ml Na2HPO4, 0.24 mg/ml KH2PO4 (pH 7.4), 1 mg/ml BSA) containing 1 mg of a fluorescein secondary antibody (ALEXA FLUOR™546 goat anti-rat IgG (H+L) conjugate (Molecular Probe)), and then incubated on ice for 30 min while shielding from light. During their respective incubation for 30 min, the cells and the antibody solution were occasionally mixed by a turning-over method for thorough mixing. The cells were collected, washed twice with PBS, suspended in 40 ml of PBS, and then observed with a fluorescence microscope (FIG. 2).

As a result, expression of Pir1-HA-Gma12 fusion protein on the cell surface layer was confirmed for the strain W303-YEp352GAP-II(PIR1-HA-gma12).

Example 5: Measurement of galactosyltransferase activity

Galactosyltransferase activity was measured by referring to Yoko-O et al's method (Yoko-O, Eur. J. Biochem., 257, 630-637 (1998)). As an enzyme source, the yeast intact cell itself (W303-YEp352GAP-II(PIR1-HA-gma12)) prepared in Example 3 was used. As an acceptor substrate, PA-mannobiose was used; as a donor substrate, UDP- galactose was used. A reaction solution of 50 ml (100 mM HEPES (pH7.2), 1 mM MnCl₂, 5 mM UDP-galactose, 300 pmol PA-mannobiose) was prepared to contain 11 ml of a cell suspension, followed by incubation at 37° C. for 5 hours. The cell suspension used herein was prepared by collecting 1 ml of a culture solution with OD600=6, washing twice with Wash Buffer (10 mM Tris-HCl (pH 8), 1 mM PMSF), and suspending in 11 ml of Wash Buffer (10 mM Tris-HCl (pH 8), 1 mM PMSF). Then, 30 ml of ice-cooled water was added to the reaction solution. The precipitated cells were removed by centrifugation at 3,000 rpm for 3 min, the supernatant with a molecular weight of 10,000 or more was removed with an Ultra Free (0.22 mm), and then mannobiose and galactosylmannobiose were measured with HPLC. An AMIDE-80™ column (TSK gel AMIDE-80™,TOSOH, 0.46cm in diameter ×25 cm in length) was used for HPLC. A mixture A containing 200 mM acetic acid-triethylamine buffer (pH 7.0) and acetonitrile (10:90), and a mixture B containing 200 mM acetic acid-triethylamine buffer (pH 7.0) and acetonitrile (60:40) were prepared. The column had been previously equilibrated by running the solvent A through the column at a flow rate of 1.0 ml/min. Immediately after injection of samples, the proportion of the solvent B was raised linearly for 60 min up to 100%, so that PA-oligosaccharide was eluted.

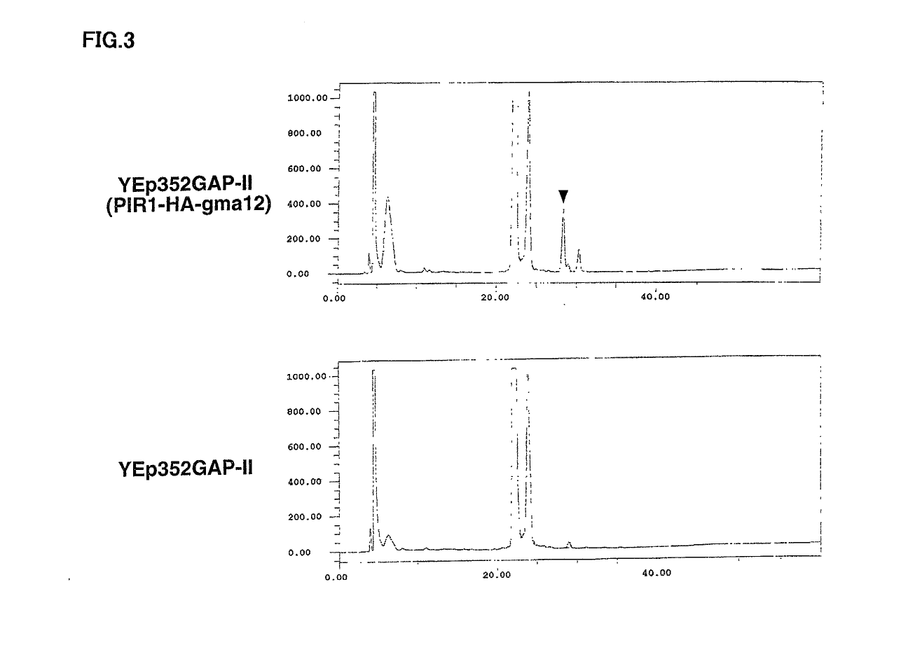

As a result, a peak of the enzyme product was detected, that is, galactosyltransferase activity could be confirmed, only for the strain W303-YEp352GAP-II (PIR1-HA-gma12) that can express a fusion protein of Pir1-HA-gma12 (FIG. 3). No galactosyltransferase activity was detected for the strain W303-YEp352GAP-II that expresses no fusion gene.

Example 6: Preparation of PIR1-HA-FUT6 fusion gene, fusion gene expression vector, and yeast transformant containing the plasmid

A plasmid pBS(SK-)/FT6H1.3 (provided by Dr. Narumatsu of Soka University) containing the amino acid coding region of a FUT6 gene, which is a human a-1,3-FucT (DB name:GenBank; Accession No: L01698) □Weston, J. Biol. Chem., 267, 24575-24585 (1992)), was used as a template. A primer previously containing an Sall site on the N-terminal side and an XhoI site on the C-terminal side was designed to enable amplification except for a transmembrane region located on the N-terminal side of an FUT6 protein. Primers having the following base sequences were used.

| | | |
|---|---|---|
| Forward primer: | 5′-CCCGTCGACAATCCTATCTGCGTGTGTCTCAAGAC-3′ | SEQ ID NO: 7 |
| Reverse primer: | 5′-CCCCTCGAGTCAGGTGAACCAAGCCGCTATGCCGC-3′ | SEQ ID NO: 8 |

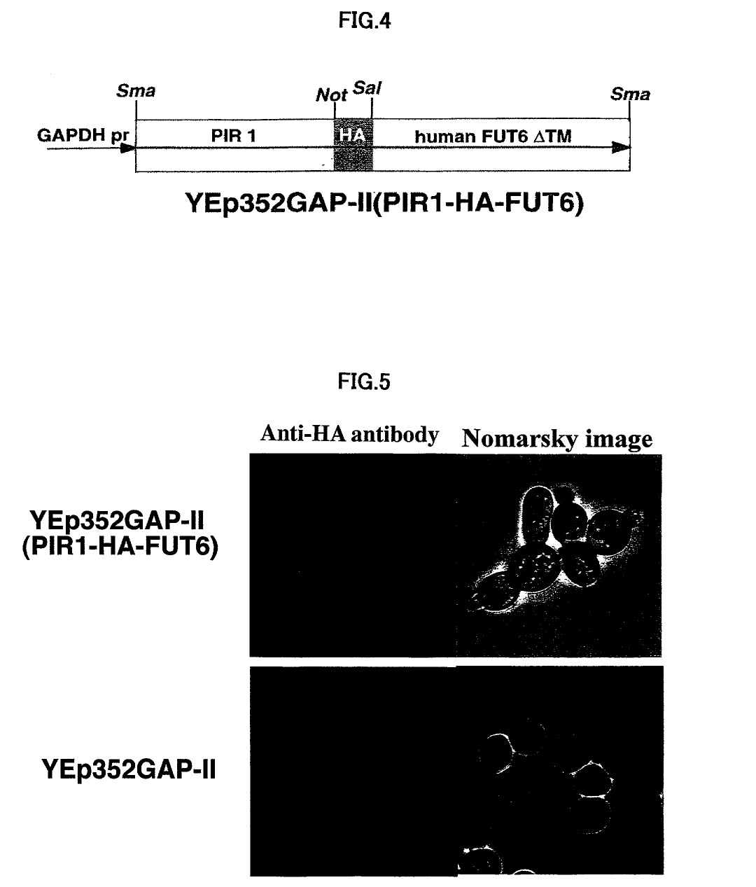

The underlined portion in the base sequence of the forward primer indicates the Sall site; that of the reverse primer indicates the XhoI site. The composition of a reaction solution and the reaction conditions employed for PCR followed Table 2 of Example 1 and the reaction conditions for Example 1, respectively. The amplified fragment of approximately 1 kb was inserted in-frame to the SalI-XhoI site of pBSII(PIR1-HA-gma12), thereby constructing pBSII(PIR1-HA-FUT6)(pAB7). The PIR1-HA-FUT6 portion was cleaved with SacI-XhoI from the pBSII(PIR1-HA-FUT6), blunt-ended with Blunting high (TOYOBO), and then inserted into an Smal site of an expression vector YEp352GAP-II (provided by Nakayama of the inventors'laboratory), thereby constructing YEp352GAP-II (PIR1-HA-FUT6)(pAB9) (FIG. 4).

The expression vector YEp352GAP-II(PIR1-HA-FUT6) was transformed to a yeast strain W303-1A (ura3, leu2, his3, trp1, ade2) (Kainuma et al., Glycobiology Vol 9:133-141 (1999)), thereby obtaining a strain W303- YEp352GAP-II(PIR1-HA-FUT6).

In addition, the strain W303- YEp352GAP-II(PIR1-HA-FUT6) was deposited under the Accession No. FERM BP-7797 at the International Patent Organism Depository, National Institute of Advanced Industrial Science and Technology on Nov. 16, 2000.

Example 7: Expression of Pir1-HA-FUT6 fusion protein within yeast cell

The transformant obtained in Example 6 was examined by the indirect immunofluorescent technique to ascertain whether a fusion gene was expressed within a cell and a fusion protein was presented on the surface layer of the yeast cell.

First, the above transformant strain W303-YEp352GAP-II(PIR1-HA-FUT6) and a control strain W303-YEpGAP-II, which had been transformed from a strain W303-1A using YEp352GAP-Il, were cultured in 5 ml of an SD (-uracil) liquid medium to OD600=5 (for approximately 30 hours). Then, 1 ml of the culture solution was collected, and then the cells were washed with PBS [8 mg/ml NaCl, 0.2 mg/ml KCl, 1.44 mg/ml Na₂HPO4, 0.24 mg/ml KH₂PO₄ (pH7.4)]. The cells were collected, suspended in 250 ml of a PBS solution (8 mg/ml NaCl, 0.2 mg/ml KCl, 1.44 mg/ml Na₂HPO₄, 0.24 mg/ml KH₂PO4 (pH 7.4), 1 mg/ml BSA) containing 1 mg of HA antibody [Anti-HA High Affinity (Roche)], and then incubated on ice for 30 min. The cells of each strain were collected respectively and washed once with a PBS solution. Subsequently, the cells were suspended in 250ml of a PBS solution (8 mg/ml NaCl, 0.2 mg/ml KCl, 1.44 mg/ml Na₂HPO4, 0.24 mg/ml KH₂PO4 (pH7.4), 1 mg/ml BSA) containing 1 mg of a labeled secondary antibody (Alexa FLUOR™ 546 goat anti-rat IgG (H+L) conjugate (Molecular Probe)), and then incubated on ice for 30 min while shielding from light. During their respective incubation for 30 min, the cells and the antibody solution were mixed occasionally by a turning-over method for thorough mixing. The cells were collected, washed twice with PBS, suspended in 40 ml of PBS, and then observed with a fluorescence microscope.

As a result, expression of Pir1-HA-FUT6 fusion protein on the cell surface layer was confirmed for the strain W303-YEp352GAP-II(PIR1-HA-FUT6) (FIG. 5).

Example 8: Measurement of fucosyltransferase activity

Fucosyltransferase activity was measured by referring to GLYCOBIOLOGY Experimental Protocol (ed. Taniguchi et al., 156-159 (1996)). As an enzyme source, a solution was prepared by disrupting yeast cells (W303-YEp352GAP-II(PIR1-HA-FUT6), prepared in Example 6) with glass beads in Wash Buffer (10 mM Tris-HCl (pH8), 1 mM PMSF). PA-Lacto-N-neotetraose was used as an acceptor substrate; GDP-fucose was used as a donor substrate. 5.5μl of the cell disruption solution was added to 4.5μ of a reaction solution (50mM Cacodylate buffer (pH 6.8), 5mM ATP, 25 mM MnCl2, 0.075 mM GDP-fucose 0.075 mM PA-Lacto-N-neotetraose), followed by incubation at 37□or 5 hours. A cell suspension used herein was a solution containing disrupted cells which had been prepared by collecting 0.25ml of the culture solution with OD600=6, washing twice with Wash Buffer (10 mM Tris-HCl (pH 8), 1 mM PMSF) and then crushing with glass beads. Next, to stop reaction, incubation was performed at 98° C. for 3 min, and then 40ml of ice-cooled water was added to the reaction solution. The precipitated cells were removed by centrifugation at 3,000 rpm for 3 min, and then the supernatant with a molecular weight of 10,000 or more was removed with an ULTRA FREE™ (0.22mm). Subsequently, Lacto-N-neotetraose and Lacto-N-fucopentaose were measured with HPLC. An Amide-80 column (TSK gel Amide-80, TOSOH, 0.46cm in diameter ×25 cm in length) was used for HPLC. A mixture A containing 200 mM acetic acid-triethylamine buffer (pH 7.0) and acetonitrile (10: 90), and a mixture B containing 200mM acetic acid-triethylamine buffer (pH 7.0) and acetonitrile (60:40) were prepared. The column had been previously equilibrated by running the solvent A through the column at a flow rate of 1.0 ml/min. Immediately after injection of samples, the proportion of the solvent B was raised linearly for 60 min to 100%, so that PA-oligosaccharide was eluted.

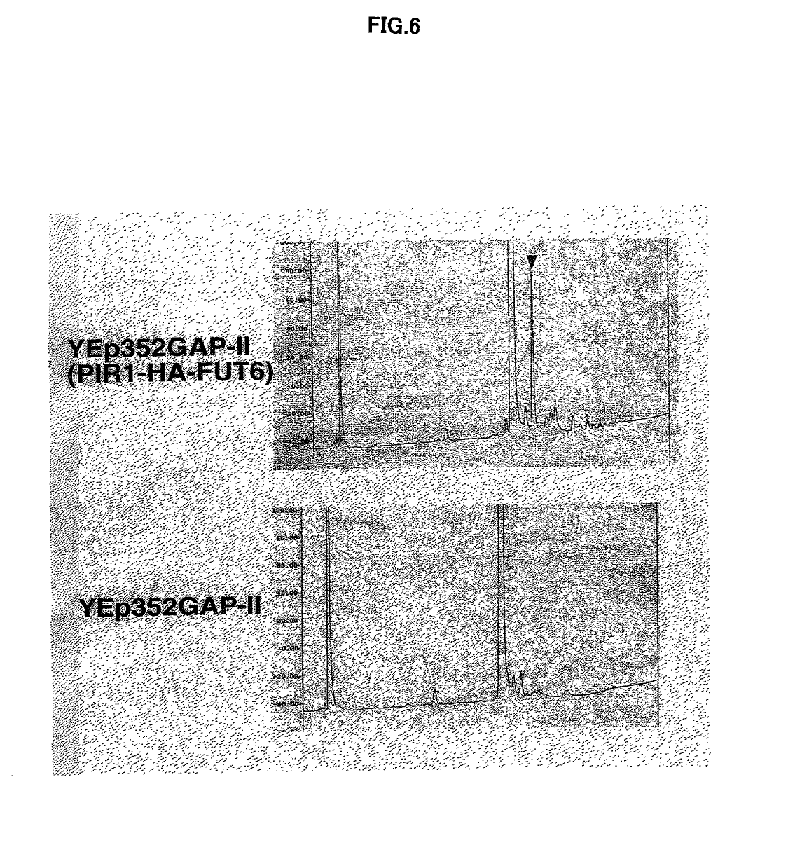

As a result, a peak of the enzyme product was detected, that is, fucosyltransferase activity could be confirmed, only for the strain W303-YEp352GAP-II (PIR1-HA-FUT6) that can express a fusion protein of Pir1-HA-FUT6 (FIG. 6). No fucosyltransferase activity was detected for the strain W303-YEp352GAP-II that expresses no fusion gene.

Example 9: Preparation of PIR1-HA-KRE2 fusion gene and fusion gene expression vector

A KRE2 gene was isolated by PCR using the genome obtained from a budding yeast strain W303-1A (ura3, leu2, his3, trp1, ade2) (Kainuma et al., Glycobiology Vol. 9, 133-141 (1999)) as a template.

Primers were designed based on a base sequence registered on a database (DB name: GenBank; Accession No: X62647). At this time, the primers previously containing an SalI site on the N-terminal side and an XhoI site on the C-terminal side were designed to enable amplification without the transmembrane region located on the N-terminal side. Primers having the following base sequences were used.

| | | |
|---|---|---|
| Forward primer | 5′-GGGGGGTCGACAGCAATATATTCCGAGTTCCATCTCCGC-3′ | SEQ ID NO: 9 |
| Reverse primer | 5′-GGGGGCTCGAGCTACTCACGGAATTTTTTCCAGTTTTTTGGC-3′ | SEQ ID NO: 10 |

Here, the underlined portion in the base sequence of the forward primer indicates the Sall site; that of the reverse primer indicates the XhoI site. Thus the designed primers were synthesized by standard techniques.

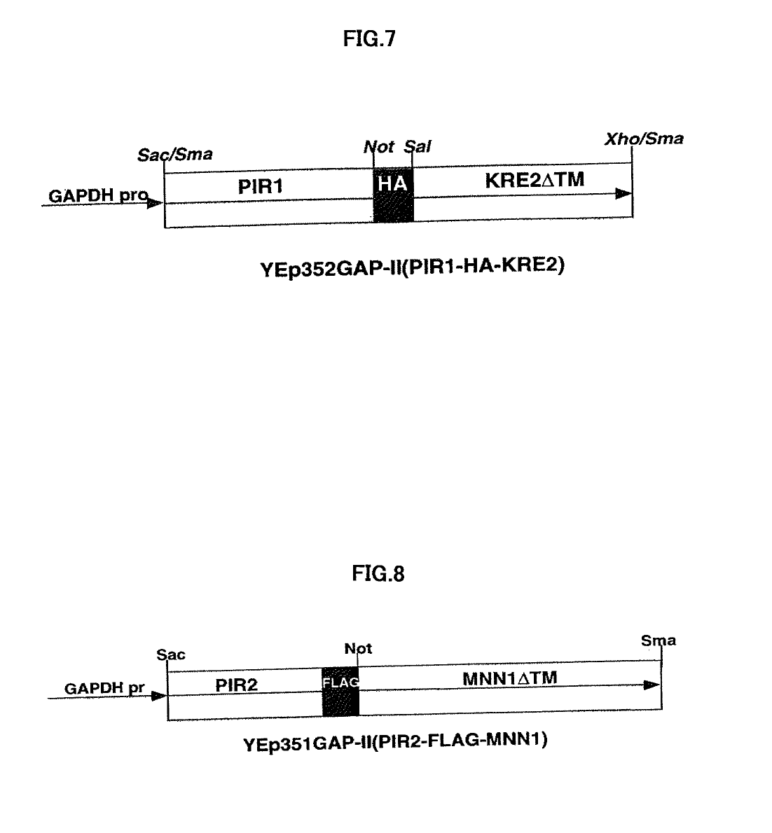

The composition of a reaction solution and the reaction conditions employed for PCR followed Table 1 of Example 1 and the reaction conditions of Example 1, respectively. The amplified fragment of approximately 1 kb was inserted in-frame to the Salil- XhoI site of pBSII(PIR1-HA-gma12), thereby constructing pBSII(PIR1 -HA-KRE2)(pAB27). The PIR1-HA-KRE2 portion was cleaved with SacI-XhoI from the pBSII(PIR1-HA-KRE2), blunt-ended with Blunting high (TOYOBO), and then inserted into an Smal site of an expression vector YEp352GAPII(provided by Nakayama of the inventors laboratory), thereby constructing YEp352GAP-II(PIR1-HA-KRE2)(pAB30) (FIG. 7).

Example 10: Construction of fusion protein composed of budding yeast Pir2 and FLAG

A PIR2 gene was isolated by PCR using the genome obtained from a budding yeast strain W303-1A (ura3, leu2, his3, trp1, ade2) (Kainuma et al., Glycobiology Vol. 9, 133-141 (1999)) as a template.

Primers were designed based on a base sequence registered on a database (DB name: GenBank; Accession No: D13741). At this time, the primers previously containing an SacI site at the N-terminal portion and the sequence of FLAG epitope tag sequence containing an NotI site at the C-terminal portion were designed to enable easy cleavage of a portion encoding a protein with a restriction enzyme and to enable a epitope tag sequence to be added to the C-terminal portion of a Pir2 protein. The base sequences of each primer were as follows.

Forward primer

5′-GGGGGGAGCTCATGCAATACAAAAAGACTTTGGTTGCC-3′
SEQ ID NO: 11

Reverse primer

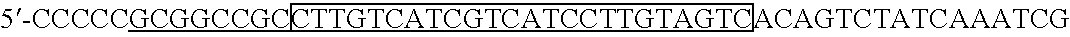

ATAGCTTCCAAGTGG-3′
SEQ ID NO: 12

The underlined portion in the base sequence of the forward primer indicates the SacI site; that of the reverse primer indicates the NotI site. The box portion indicates a sequence of a FLAG epitope tag. Thus the designed primers were synthesized by standard techniques.

The composition of a reaction solution and the reaction conditions employed for PCR followed Table 1 of Example 1 and the reaction conditions of Example 1, respectively. The amplified fragment of approximately 1 kb was cleaved with SacI-NotI, and then inserted to the SacI-NotI site of pBluescript II SK(-) (Stratagene), thereby constructing pBSII(PIR2-FLAG)(pAB22).

Example 11: Preparation of PIR2-FLAG-MNN1 fusion gene and fusion gene expression vector

An MNN1 gene was isolated by PCR using the genome obtained from a budding yeast strain W303-1A (ura3, leu2, his3, trp1, ade2) (Kainuma et al., Glycobiology Vol 9, 133-141 (1999)) as a template.

Primers were designed based on a base sequence registered on a database (DB name: GenBank; Accession No: L23753). At this time, the primers previously containing an NotI site on the N-terminal side and an Smal site on the C-terminal side were designed to enable amplification without the transmembrane region located on the N- terminal side. Primers having the following base sequences were used.

| | | |
|---|---|---|
| Forward primer | 5′-GGGGGGCGGCCGCAAATGATGCGCTTATACGATCAAGCAATGTAAACAG-3′ | SEQ ID NO: 13 |
| Reverse primer | 5′-GGGGGCCCGGGCTAGCTTTGTTCGTGTCTAGAATTTTC-3′ | SEQ ID NO: 14 |

The underlined portion in the base sequence of the forward primer indicates the NotI site; that of the reverse primer indicates the SmaI site. Thus, the designed primers were synthesized by standard techniques.

The composition of a reaction solution and the reaction conditions employed for PCR followed Table 1 of Example 1 and the reaction conditions of Example 1, except that the time for elongation reaction was changed from 1 to 2 min. The amplified fragment of approximately 2 kb was inserted in-frame to the NotI-SmaI site of pBSll(PIR2- FLAG)(pAB22), thereby constructing pBSII(PIR2-FLAG-MNN1)(pAB28). The PIR2-FLAG-MNN1 portion was cleaved with Sacl-Smal from the pBSII(PIR2-FLAG-MNN1), and then inserted into an SacI-Smal site of an expression vector YEp352GAP-II(provided by Nakayama of the inventors laboratory), thereby constructing YEp352GAP-II(PIR2-FLAG- MNN1) (pAB29). Further, to construct a plasmid having a leucine marker, a BgII fragment containing a promoter region, PIR2-FLAG-MNN1 and a terminator region was cleaved from Yep352GAP-II(PIR2-FLAG-MNN1), and then inserted into a BglI site of self amplification vector YEp351 (Hill et al., YEAST, 2, 163-167 (1986)), thereby constructing YEp351GAP-II (PIR2-FLAG-MNN1)(pAB31) (FIG. 8).

Example 12: Construction of yeast transformant containing expression vector YEp352GAP-II (PIR1-HA-KRE2) and YEp351GAP-II(PIR2-FLAG-MNN1), and expression of both fusion proteins in yeast cells

The expression vectors YEp352GAP-II(PIR1-HA-KRE2) and YEp351GAP-II(PIR2-FLAG-MNN1) were transformed simultaneously to a yeast strain W303-1A (ura3, leu2, his3, trp1, ade2)□Kainuma et al., Glycobiology, 9□133-141(1999) □, thereby obtaining a strain W303-YEp352GAP-II(PIR1-HA-KRE2), YEp351GAP-II(PIR2-FLAG-MNN1). The strain W303-YEp352GAP-II(PIR1-HA-KRE2), YEp351GAP-II(PIR2- FLAG-MNN1) was deposited under the Accession No. FERM BP-7789 at the International Patent Organism Depository, National Institute of Advanced Industrial Science and Technology on Jun. 20, 2001.

The obtained transformant was subjected to Western blotting to examine if fusion genes were co-expressed in a cell and fusion proteins were co-localized on the yeast cell surface layer. PIR protein was covalently attached to a cell wall by an alkali-sensitive linkage, suggesting that a fusion protein with PIR would be freed by a mild alkali treatment of the cell wall fraction.

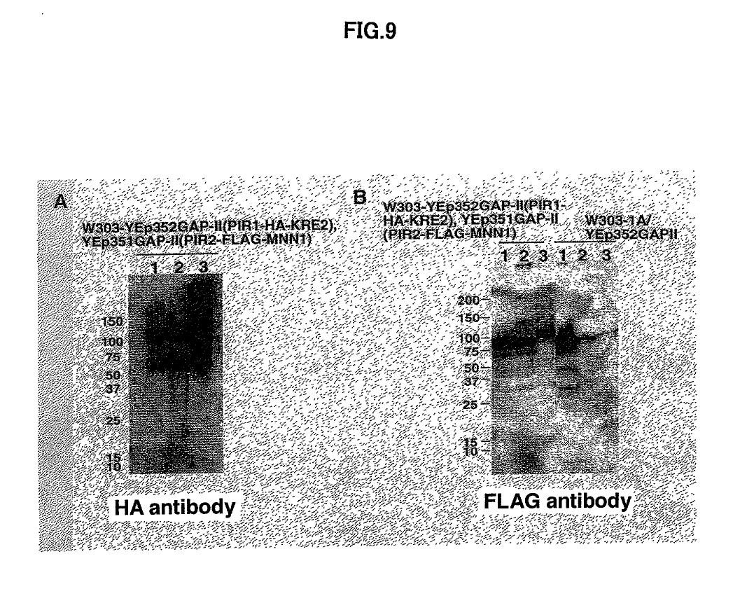

First, the above transformant strain W303-YEp352GAP-II(PIR1-HA-KRE2), YEp351GAP-ll(PIR2-FLAG-MNN1) and a control strain W303-YEp352GAP-II, which had been transformed from a strain W303-1A using YEp352GAP-II, were cultured in 5 ml each of an SD (-uracil, -leucine) and an SD(-uracil) liquid media to OD600=5 (for approximately 48 hours). Then, the cells were collected and washed with Wash Buffer (lOmM Tris-HCl (pH8.0), ImM PMSF). Glass beads were added to the cell suspension and Voltex was applied at 4° C. for 15 min, so that the cells were disrupted. The solution containing the disrupted cells was separated into supernatant (Lane 1 of FIG. 9A and B) and pellet. The pellet was washed three times with Wash Buffer (10 mM Tris-HCl (pH8.0), 1 mM PMSF), suspended in 100mI of Laemmli Buffer (4% SDS, 20% glycerol, 0.12M Tris-HCl (pH6.6), 8M urea, 2% b-ME□, and then boiled at 100) for 10 min. This sample was centrifuged and further separated into supernatant (Lane 2 of FIG. 9A and B) and pellet fraction. The pellet fraction was washed three times with Na-acetate Buffer (pH 5.5) [M Na-acetate], and then incubated with 100 ml of a mild alkali solution [30mM NaOH] at 4° C. for 15 hours. The suspension with the mild alkali solution was centrifuged, so that supernatant was collected (Lane 3 of FIG. 9A and B). The collected samples were subjected to SDS-PAGE, and then Western blotting. At this time, primary antibodies used herein were HA antibody □MONOCLONAL ANTIBODY,HA. 11 (CONVANCE) and FLAG antibody-ANTI-FLAG M2 Monoclonal Antibody (SIGMA); and a secondary antibody used herein was anti-mouse IgG-HRP-Anti-Mouse IgG (H&L) HRP-Linked Antibody ( Cell Signaling TECHNOLOGY). To perform immuno-staining with 2 types of antibodies, HA antibody and FLAG antibody, two membranes to which the same protein solution had been blotted were prepared, and then immuno-staining was performed separately with the 2 types of antibodies. Thus, a specific band was detected only for a strain expressing the fusion protein when the cell wall fraction was treated with mild alkali. This result reveals that Pir1-HA-Kre2 fusion protein and Pir2-FLAG-Mnn1 fusion protein were localized simultaneously on a cell wall with a binding pattern (to a cell wall) representing the characteristics of PIR.

Example 13: Measurement of sequential transfer reaction of mannose

Mannosyltransferase activity was measured by referring to Lussier et al's method (Lussier et al., JBC., 271, 11001-11008 (1996)). As an enzyme source, the yeast intact cell itself (W303- YEp352GAP-II(PIR1-HA-KRE2), YEp351GAP-II(PIR2-FLAG- MNN1)) prepared in Example 12 was used. A control strain used herein was W303- YEp352GAP-II. As an acceptor substrate, PA-mannobiose was used; as a donor substrate, GDP-mannose was used. A reaction solution 50ml (100 mM HEPES(pH7.2), 1 mM MnCl2,5 mM GDP-mannose, 300 pmol PA-mannobiose) was prepared to contain 20 ml of a cell suspension, followed by incubation at 37 °C. for 3 hours. The cell suspension used herein was prepared by collecting 1 ml of a culture solution with OD600=4, washing twice with Wash Buffer (10 mM Tris-HCl(pH8), 1 mM PMSF), and then suspending in 20 ml of Wash Buffer. Then, 50 ml of ice-cooled water was added to the reaction solution. The precipitated cells were removed by centrifugation at 3,000 rpm for 3 min, and then the supernatant with a molecular weight of 10,000 or more was removed with an Ultra Free (0.22 mm). Then, mannobiose (disaccharide), mannotriose (trisaccharide) and mannotetraose (tetraose) were detected with HPLC. Amide-80 column (TSK gel An Amide-80, TOSOH, 0.46 cm in diameter ×25 cm in length) was used for HPLC. A mixture A containing 200 mM acetic acid-triethylamine buffer (pH 7.0) and acetonitrile (10:90), and a mixture B containing 200 mM acetic acid-triethylamine buffer (pH 7.0) and acetonitrile (60:40) were prepared. The column had been previously equilibrated by running the solvent A through the column at a flow rate of 1.0 ml/min. Immediately after injection of samples, the proportion of the solvent B was raised linearly for 60 min to 100%, so that PA-oligosaccharide was eluted.

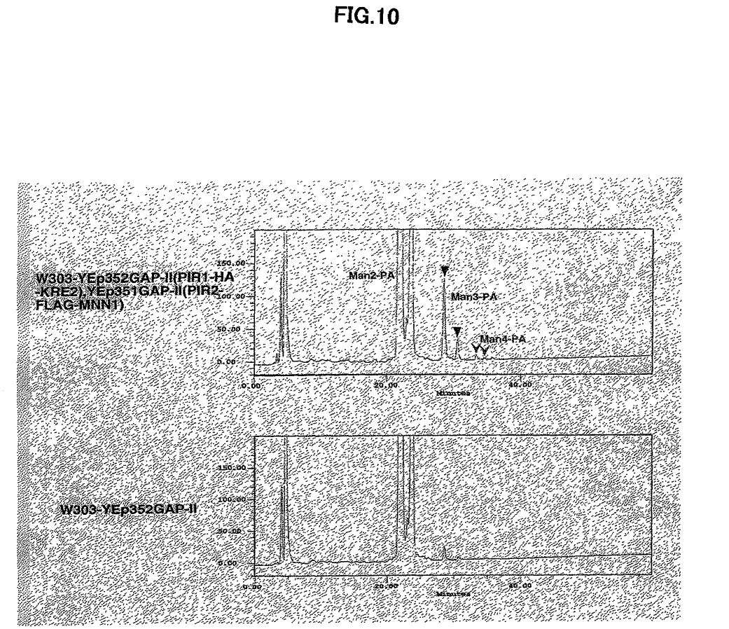

As a result, peaks indicating trisaccharide (mannotriose) and tetraose (mannotetraose) were detected only for the strain W303- YEp352GAP-II(PIR1-HA-KRE2), YEp351GAP-II(PIR2-FLAG-MNN1) that can simultaneously express both fusion proteins, Pir1-HA-Kre2 and Pir2-FLAG-Mnn1 (FIG. 10). Regarding the strain W303-YEp352GAP-II that expresses no fusion gene, almost no peak indicating trisaccharide (mannotriose) and tetraose (mannotetraose) was observed (FIG. 10).

These results suggest that integration of PIR1 and PIR2 as anchor proteins to a cell wall onto the N-terminal side of a useful protein enables presentation of the useful protein on the yeast cell surface layer. Moreover, the results also suggest that simultaneous expression of the above fusion proteins can easily cause a complex sequential reaction of enzyme on a yeast cell surface layer.

Effect of the invention

The present invention enables immobilization of a useful protein, such as a glycosyltransferase, onto the surface of a yeast cell without deteriorating its enzyme activity, so that the invention can provide the immobilized protein as an immobilized enzyme. Since a process for purifying enzymes and a process for immobilizing enzymes to beads can be omitted, immobilized enzyme can be produced very easily and in large quantities.

All the documents cited in this specification are incorporated into the specification as references in their entirety.

What is claimed is:

1. A chimeric nucleic acid comprising a first domain comprising a yeast Pir (protein internal repeat) cell wall protein coding sequence and a second domain comprising a peptide or a polypeptide coding sequence, wherein the yeast cell wall protein is capable of being localized or immobilized on a yeast cell wall.

2. The chimeric nucleic acid of claim 1, wherein the yeast cell wall protein comprises a Pir (protein internal repeat) motif coding sequence.

3. The chimeric nucleic acid of claim 2, wherein the Pir (protein internal repeat) protein motif comprises an amino acid sequence as set forth by SEQ ID NO: 1 or SEQ ID NO:2.

4. The chimeric nucleic acid of claim 2, wherein the Pir (protein internal repeat) protein motif comprises a protein comprising an amino acid sequence derived from an amino acid sequence as set forth by SEQ NO: 1 by deletion, replacement, or addition of one or more amino acids of SEQ NO: 1, wherein the Pir (protein internal repeat) protein motif is capable of being localized or immobilized on a yeast cell wall.

5. The chimeric nucleic acid of claim 1, wherein the polypeptide is an enzyme.

6. The chimeric nucleic acid of claim 1, wherein the enzyme is a glycosyltransferase.

7. The chimeric nucleic acid of claim 1, wherein the Pir (protein internal repeat) protein motif coding sequence is located 5′ to the peptide or a polypeptide coding sequence.

8. An expression cassette comprising a chimeric nucleic acid comprising a first domain comprising a Pir (protein internal repeat) protein motif coding sequence and a second domain comprising a peptide or a polypeptide coding sequence.

9. The expression cassette of claim 8 comprising an expression vector.

10. The expression cassette of claim 9, wherein the expression vector comprises a yeast expression vector.

11. A host cell comprising an expression cassette comprising a chimeric nucleic acid comprising a first domain comprising a Pir (protein internal repeat) protein motif coding sequence and a second domain comprising a peptide or a polypeptide coding sequence.

12. The host cell of claim 11 comprising a yeast cell.

13. The host cell of claim 11 comprising a yeast cell wall.

14. An expression vector comprising a fusion gene comprising a nucleic acid encoding a useful protein downstream of a nucleic acid encoding a yeast cell wall protein selected from the group consisting of (a) a protein having an amino acid sequence represented by SEQ ID NO:1, and (b) a protein comprising an amino acid derived from an amino acid sequence as set forth by SEQ NO:1 by deletion, replacement, or addition of one or more amino acids of SEQ NO:1, wherein yeast cell wall protein is capable of being localized or immobilized on a yeast cell wall.

15. The expression vector of claim 14, wherein the useful protein is a glycosyltransferase protein.

16. A transformant yeast transformed by an expression vector, wherein the expression vector comprises a chimeric nucleic acid comprising a nucleic acid encoding a useful protein downstream of a nucleic acid encoding a yeast cell wall protein selected from the group consisting of (a) a protein having an amino acid sequence represented by SEQ ID NO:1, and (b) a protein comprising an amino acid derived from an amino acid sequence as set forth by SEQ NO:1 by deletion, replacement, or addition of one or more amino acids of SEQ NO: 1, wherein yeast cell wall protein is capable of being localized or immobilized on a yeast cell wall.

17. A chimeric polypeptide comprising a first domain comprising a yeast cell wall protein and a second domain comprising a peptide or a polypeptide of interest, wherein the yeast cell wall protein is capable of being localized or immobilized on a yeast cell wall.

18. A particle comprising a chimeric polypeptide comprising a first domain comprising a yeast cell wall protein and a second domain comprising a peptide or a polypeptide of interest, wherein the yeast cell wall protein is capable of being localized or immobilized on a yeast cell wall component, and a yeast cell wall component.

19. The particle of claim 18, wherein the particle is a resin.

20. A solid support comprising a chimeric polypeptide comprising a first domain comprising a yeast cell wall protein and a second domain comprising a peptide or a polypeptide of interest, wherein the yeast cell wall protein is capable of being localized or immobilized on a yeast cell wall component, and a yeast cell wall component.

21. The solid support of claim 20, wherein the solid support comprises a tube, a fiber, a plate or a filter.

22. A method for producing an immobilized polypeptide comprising the following steps:
(a) providing an expression vector, wherein the expression vector comprises a chimeric nucleic acid encoding a fusion polypeptide, wherein the chimeric nucleic acid comprises a nucleic acid encoding a useful protein downstream of a nucleic acid encoding a yeast cell wall protein selected from the group consisting of (a) a protein having an amino acid sequence represented by SEQ ID NO:1, and (b) a protein comprising an amino acid derived from an amino acid sequence as set forth by SEQ NO:1 by deletion, replacement, or addition of one or more amino acids of SEQ NO:1, wherein yeast cell wall protein is capable of being localized or immobilized on a yeast cell wall;
(b) transforming a microorganism comprising a yeast cell wall with the expression vector of step (a);
(b) culturing the transformant microorganism of step (b) and expressing the fusion polypeptide on a surface layer of the yeast cell wall, thereby producing an immobilized polypeptide.

23. The method of claim 22, wherein the useful protein is a glycosyltransferase protein.

24. The method of claim 22, wherein the microorganism comprises a yeast.

25. An immobilized enzyme obtained by the method of claim 22.

26. The immobilized enzyme of claim 25, wherein the enzyme is a glycosyltransferase.

27. A method for producing a sugar chain or a sugar comprising use of an immobilized enzyme as set forth in claim 22.

28. A method for producing an immobilized enzyme comprising culturing the host cell of claim 12 and obtaining a yeast comprising a useful protein immobilized on its cell wall.

29. An immobilized enzyme obtained by the method of claim 28.

30. The immobilized enzyme of claim 29, wherein the enzyme immobilized is a glycosyltransferase.

31. A method for producing a sugar chain or a sugar which employs the immobilized enzyme of claim 29.

32. A transformant yeast which is transformed by allowing the yeast to comprise an expression cassette as set forth in claim 8 or an expression vector as set forth in claim 14.

33. A method for producing an immobilized enzyme which comprises the steps of:
(a) culturing the transformant yeast of claim 32,
(b) expressing chimeric polypeptides on the surface layer a cell wall of the transfonnant yeast, and
(c) isolating a transformant yeast that expresses a chimeric polypeptide immobilized on the cell wall.

34. An immobilized enzyme obtained by the method of claim 33.

35. The immobilized enzyme of claim 34, wherein the enzyme immobilized is a glycosyltransferase.

36. A method for producing a sugar chain or a sugar, wherein the method comprises sequentially converting a sugar chain or a sugar using an immobilized enzyme as set forth in claim 34.

37. A chimeric nucleic acid comprising a first domain comprising a yeast cell wall protein coding sequence and a second domain comprising an enzyme coding sequence, wherein the yeast cell wall protein is capable of being localized or immobilized on a yeast cell wall and the enzyme is selected from the group consisting of a fucosyltransferase, a Lacto-N-fucopentaose, a galactosyltransferase, and a glucosyltransferase.

38. A chimeric polypeptide comprising a first domain comprising a yeast cell wall protein and a second domain comprising an enzyme, wherein the yeast cell wall protein is capable of being localized or immobilized on a yeast cell wall and the enzyme is selected from the group consisting of a fucosyltransferase, a Lacto-N-fucopentaose, a galactosyltransferase, and a glucosyltransferase.


SEQUENCE LISTING (14 sequences)

SEQ ID NO: 1 - protein, 341 aa, Saccharomyces cerevisiae
MQYKKSLVASALVATSLAAYAPKDPWSTLTPSATYKGGITDYSSTFGIAVEPIATTASSKAKRAAAISQIGDGQIQATTKTTAAAVSQIGDGQIQATTKTKAAAVSQIGDGQIQATTKTTSAKTTAAAVSQIGDGQIQATTKTKAAAVSQIGDGQIQATTKTTAAAVSQIGDGQIQATTKTTAAAVSQIGDGQIQATTNTTVAPVSQITDGQIQATTLTSATIIPSPAPAPITNGTDPVTAETCKSSGTLEMNLKGGILTDGKGRIGSIVANRQFQFDGPPPQAGAIYAAGWSITPEGNLAIGDQDTFYQCLSGNFYNLYDEHIGTQCNAVHLQAIDLLNC

SEQ ID NO: 2 - protein, 413 aa, Saccharomyces cerevisiae
MQYKKTLVASALAATTLAAYAPSEPWSTLTPTATYSGGVTDYASTFGIAVQPISTTSSASSAATTASSKAKRAASQIGDGQVQAATTTASVSTKSTAAAVSQIGDGQIQATTKTTAAAVSQIGDGQIQATTKTTSAKTTAAAVSQISDGQIQATTTTLAPKSTAAAVSQIGDGQVQATTTTLAPKSTAAAVSQIGDGQVQATTKTTAAAVSQIGDGQVQATTKTTAAAVSQIGDGQVQATTKTTAAAVSQIGDGQVQATTKTTAAAVSQITDGQVQATTKTTQAASQVSDGQVQATTATSASAAATSTDPVDAVSCKTSGTLEMNLKGGILTDGKGRIGSIVANRQFQFDGPPPQAGAIYAAGWSITPDGNLAIGDNDVFYQCLSGTFYNLYDEHIGSQCTPVHLEAIDLIDC

SEQ ID NO: 3 - DNA, 44 bp, Artificial Sequence
  Synthetic primer
ggggggagctcatgcaatacaaaaaatcattagttgcctccgcc

SEQ ID NO: 4 - DNA, 31 bp, Artificial Sequence
  Synthetic primer
cccccgcggccgcacagtgcaaatcgatagc

SEQ ID NO: 5 - DNA, 35 bp, Artificial Sequence
  Synthetic primer
ggggggcggccgcatacccatacgatgttcctgac

SEQ ID NO: 6 - DNA, 47 bp, Artificial Sequence
  Synthetic primer
ggggcccgggctaggatgatggtttcaaaagattttgaatatgatcc

SEQ ID NO: 7 - DNA, 35 bp, Artificial Sequence
  Synthetic primer
cccgtcgacaatcctatctgcgtgtgtctcaagac

SEQ ID NO: 8 - DNA, 35 bp, Artificial Sequence
  Synthetic primer
cccctcgagtcaggtgaaccaagccgctatgccgc

SEQ ID NO: 9 - DNA, 39 bp, Artificial Sequence
  Synthetic primer
ggggggtcgacagcaatatattccgagttccatctccgc

SEQ ID NO: 10 - DNA, 42 bp, Artificial Sequence
  Synthetic primer
gggggctcgagctactcacggaattttttccagttttttggc

SEQ ID NO: 11 - DNA, 38 bp, Artificial Sequence
  Synthetic primer
ggggggagctcatgcaatacaaaaagactttggttgcc

SEQ ID NO: 12 - DNA, 68 bp, Artificial Sequence
  Synthetic primer
cccccgcggccgccttgtcatcgtcatccttgtagtcacagtctatcaaatcgatagcttccaagtgg

SEQ ID NO: 13 - DNA, 49 bp, Artificial Sequence
  Synthetic primer
ggggggcggccgcaaatgatgcgcttatacgatcaagcaatgtaaacag

SEQ ID NO: 14 - DNA, 38 bp, Artificial Sequence
  Synthetic primer
gggggcccgggctagctttgttcgtgtctagaattttc